SEQUENCE LISTING (9 sequences)

SEQ ID NO: 1 - protein, 67 aa, Homo sapiens
AYRPSETLCGGELVDTLQFVCGDRGFYFSRPASRVSRRSRGIVEECCFRSCDLALLETYCATPAKSE

SEQ ID NO: 2 - protein, 8 aa, Homo sapiens
FRSCDLAL

SEQ ID NO: 3 - protein, 21 aa, Homo sapiens
LCGGELVDTLQFVCGDRGFYF

SEQ ID NO: 4 - protein, 21 aa, Homo sapiens
GIVEECCFRSCDLALLETYCA

SEQ ID NO: 5 - protein, 60 aa, Homo sapiens
LCGGELVDTLQFVCGDRGFYFSRPASRVSRRSRGIVEECCFRSCDLALLETYCATPAKSE

SEQ ID NO: 6 - protein, 67 aa, Homo sapiens
AYRPSETLCGGELVDTLQFVCGDRGSYFSRPASRVSRRSRGIVEECCFRSCDLALLETYCATPAKSE

SEQ ID NO: 7 - protein, 67 aa, Homo sapiens
AYRPSETLCGGELVDTLQSVCGDRGFYFSRPASRVSRRSRGIVEECCFRSCDLALLETYCATPAKSE

SEQ ID NO: 8 - protein, 67 aa, Homo sapiens
AYRPSETLCGGKLVDTLQFVCGDRGFYFSRPASRVSRRSRGIVEECCFRSCDLALLETYCATPAKSE

SEQ ID NO: 9 - protein, 67 aa, Homo sapiens
AYRPSETLCGGELVDTLQFVCGDRGFLFSRPASRVSRRSRGIVEECCFRSCDLALLETYCATPAKSE

(19) United States
(12) Patent Application Publication
(10) Pub. No.: US 2008/0279765 A1
(43) Pub. Date: Nov. 13, 2008

(54) Novel Imaging Agents for Fibrosis

(76) Inventors: Salah Chettibi, Buckinghamshire (GB); Ben Newton, Buckinghamshire (GB); Benedicte Guilbert, Buckinghamshire (GB); Helge Tolleshaug, Oslo (NO); Magne Solbakken, Oslo (NO)

Correspondence Address: GE HEALTHCARE, INC., IP DEPARTMENT, 101 CARNEGIE CENTER, PRINCETON, NJ, 08540-6231, US

(21) Appl. No.: 12/095,931

(22) PCT Filed: Dec. 7, 2006
(86) PCT No.: PCT/GB2006/004575

§ 371 Date: Jun. 3, 2008

(30) Foreign Application Priority Data
Dec. 8, 2005 (GB) 0524987.5

(51) Int. Cl. A61K 51/00; C07K 14/47; C07H 13/00; C07K 14/00; C07C 403/08; C07K 7/06; C07K 5/08; C07F 13/00; A61K 49/00

(52) U.S. Cl. 424/1.11; 530/324; 536/1.11; 530/322; 562/510; 530/329; 530/326; 530/331; 530/330; 534/14; 424/9.1

(57) ABSTRACT

The present invention provides a novel imaging agent targeting the mannose-6-phosphate (M6P) receptor suitable for the non-invasive visualization of fibrosis. A method for the preparation of the imaging agent is also provided by the invention, as well as a precursor for use in said method. The invention also provides a pharmaceutical composition comprising the imaging agent and a kit for the preparation of the pharmaceutical composition. In a further aspect, use of the imaging agent for in vivo imaging and in the preparation of a medicament for the diagnosis of a condition in which the mannose-6-phosphate receptor is upregulated is provided.

### TECHNICAL FIELD OF THE INVENTION

The present invention relates to diagnostic imaging and in particular to the diagnostic imaging of fibrosis. Diagnostic imaging agents are described which are suitable for this purpose, particularly for the diagnostic imaging of fibrosis in the liver, heart, kidneys and lungs.

### DESCRIPTION OF RELATED ART

Fibrosis is a process characterized by the excessive secretion of extracellular matrix components. This is caused by increased synthesis and decreased degradation of matrix proteins, most notably collagen types I and III, and is triggered as a response to tissue damage resulting from inflammation, infection or injury. In simple terms, fibrosis is scar tissue and forms part of all “repair” processes in tissue. However, because of ongoing inflammation, infection and repeated injury, fibrosis scar tissue builds up and does not replace “functional” cells, thus leading to abnormal organ function and eventually organ failure.

Fibrosis is one of the major, classic pathological processes in medicine. It is a key component of multiple diseases that affect millions of people worldwide including:
a) Lung diseases such as idiopathic pulmonary fibrosis (lung fibrosis of unknown origin), asthma and chronic obstructive pulmonary disease
b) Scleroderma: a heterogeneous and life threatening disease characterised by the excessive extracellular matrix deposition within connective tissue of the body (i.e. skin and visceral organs)
c) Post-surgical scarring following transplantation
d) Diabetic retinopathy and age-related macular degeneration (fibrotic diseases of the eye and leading causes of blindness)
e) Cardiovascular disease including atherosclerosis and vulnerable plaque.
f) Kidney fibrosis linked to diabetes—diabetic nephropathy and glomerulosclerosis
g) IgA nephropathy (causes of kidney failure and the need for dialysis and retransplant)
h) Cirrhosis and biliary atresia (leading causes of liver fibrosis and failure)
i) Rheumatoid arthritis
j) Autoimmune diseases such as dermatomyositis
k) Congestive heart failure
   The clinical manifestations of fibrosis vary widely. Taking the example of cirrhosis, the clinical manifestations vary from no symptoms at all, to liver failure, and are determined by both the nature and severity of the underlying liver disease as well as the extent of hepatic fibrosis. Up to 40% of patients with cirrhosis are asymptomatic and may remain so for more than a decade, but progressive deterioration is inevitable once complications develop including ascites, variceal hemorrhage or encephalopathy. Fibrosis and cirrhosis therefore represent the consequences of a sustained wound healing response to chronic liver injury from a variety of causes including viral, autoimmune, drug induced, cholestatic and metabolic diseases. The common causes of liver fibrosis and cirrhosis include immune mediated damage, genetic abnormalities, and non-alcoholic steatohepatitis (NASH), which is particularly associated with diabetes and metabolic syndrome (MS). There is a high incidence of MS in the western population. This syndrome typically occurs in individuals who are obese, have hyperlipidemia and hypertension, and often leads to the development of type II diabetes. The hepatic manifestation of metabolic syndrome is non-alcoholic fatty liver disease (NAFLD), with an estimated prevalence in the USA of 24% of the population. A fatty liver represents the less severe end of a spectrum of NAFLD that may progress to NASH and ultimately to cirrhosis of the liver. The development of fibrosis demonstrates a risk of such progression, and is presently assessed by means of a liver biopsy. However, liver biopsy causes significant discomfort, is not without risk and is costly. Furthermore, available blood tests for hepatic fibrosis are not reliable in NAFLD.

WO 00/23113 discloses a compound comprising a peptide and a carrier molecule, where the peptide targets hepatic stellate cells (HSC). Examples of target receptors presented are those specific for HSC or upregulated on HSC during disease (including fibrotic disease), e.g. platelet-derived growth factor receptor, collagen VI receptor, transforming growth factorβ receptor, interleukin-1β receptor and tumour necrosis factors receptor. In another aspect presented, the target receptor is the cation-independent mannose-6-phosphate (M6P) receptor, which has been reported to be over-expressed in liver fibrosis. Examples of suitable carriers are human serum albumin, proteins, polymeric carriers and liposomes. The compounds disclosed are primarily drug carriers which can be used to target all kinds of therapeutic agents. It is stated that the carrier is suitably larger than 5000 Da. It is also mentioned that they may be applied for the visualisation of HSC for diagnostic purposes, stating that the compounds may further comprise a diagnostic marker. However, there is no description of how a diagnostic marker might be attached to the disclosed compounds.

EP 1495769 A1 discloses glycoside-compound conjugates for use in antisense strategies, particularly in vivo antisense strategies. The conjugates comprise a glycoside linked to a compound, in which glycoside is a ligand capable of binding to a mannose-6-phosphate receptor of a muscle cell. Conjugates which are “marked” to include fluorescent, radioactive, enzymatic or molecular markers are mentioned for in vivo or in vitro diagnosis, but there is no description of how to obtain such marked compounds.

A need therefore exists for an alternative non-invasive test for the detection of fibrosis and in particular liver fibrosis.

### SUMMARY OF THE INVENTION

The present invention provides a novel imaging agent suitable for the non-invasive visualization of fibrosis. A method for the preparation of the imaging agent is also provided by the invention, as well as a precursor for use in said method. The invention also provides a pharmaceutical composition comprising the imaging agent and a kit for the preparation of the pharmaceutical composition. In a further aspect, use of the imaging agent for in vivo imaging and in the preparation of a medicament for the diagnosis of a condition in which the mannose-6-phosphate receptor is upregulated is provided.

### DETAILED DESCRIPTION OF THE INVENTION

In one aspect, the present invention provides an imaging agent comprising:

(i) a vector with affinity for the mannose-6-phosphate (M6P) receptor; and,

(ii) an imaging moiety.

wherein the imaging moiety is present either as an integral part of the vector or the imaging moiety is conjugated to the vector via a suitable chemical group.

In the context of the present invention, the term “M6P receptor” specifically relates to the extracellular domain of the cation-independent M6P receptor.

By the term “imaging agent” is meant a compound designed to target a particular physiology or pathophysiology in a mammal, and which can be detected following its administration to the mammalian body in vivo.

As stated above, in the imaging agent of the invention, the imaging moiety may be present as an integral part of the vector, e.g. one of the atoms of the vector could be ¹¹C instead of ¹²C. Alternatively, the imaging moiety may be conjugated to the vector via a suitable chemical group, e.g. a metal chelate which can complex an imaging moiety which is a metal ion. A linker may also be present linking the vector to either the suitable chemical group or directly to the imaging moiety itself. Suitable linkers of the present invention are of Formula -(L¹)ₙ- wherein:
each L¹ is independently —CO—, —CR₂—, —CR═CR—, —C≡C—, —CR₂CO₂—, —CO₂CR₂—, —NR—, —NRCO—, —CONR—, —NR(C═O)NR—, —NR(C═S)NR—, —SO₂NR—, —NRSO₂—, —CR₂OCR₂—, —CR₂SCR₂—, —CR₂NRCR₂—, a C₄₋₈ cycloheteroalkylene group, a C₄₋₈ cycloalkylene group; a C₅₋₁₂ arylene group, a C₃₋₁₂ heteroarylene group, an amino acid residue, a polyalkyleneglycol, polylactic acid or polyglycolic acid moiety;
n is an integer of value 0 to 20;
each R group is independently H or C₁₋₁₀ alkyl, C₃₋₁₀ alkylaryl, C₂₋₁₀ alkoxyalkyl, C₁₋₁₀ hydroxyalkyl, C₁₋₁₀fluoroalkyl, or 2 or more R groups, together with the atoms to which they are attached form a carbocyclic, heterocyclic, saturated or unsaturated ring.

It is envisaged that branched linker groups are also possible, i.e. the linker group -(L¹)ₙ-substituted with a further linker -(L²)ₒ-R′, wherein L², o and R′ are as defined respectively for L¹, n and R above.

Such linkers are particularly useful in the context of manipulating the biodistribution and/or excretion profiles of the imaging agent. For example, the inclusion of a linker comprising polyethylene glycol groups or acetyl groups can improve the blood residence time of the imaging agent.

By the term “amino acid” is meant an L- or D-amino acid, amino acid analogue (e.g. napthylalanine) or amino acid mimetic which may be naturally occurring or of purely synthetic origin, and may be optically pure, i.e. a single enantiomer and hence chiral, or a mixture of enantiomers. Preferably the amino acids of the present invention are optically pure.

Such linkers also have application in relation to other parts of the invention as described below. For the present application, preferred L¹ and L² groups are —CO—, —CH₂—, —NH—, —NHCO—, —CONH—, —CH₂OCH₂—, and amino acid residues.

The term “affinity” in the context of the present invention is taken to mean binding to the M6P receptor in vitro with a Kd value of less than 100 nM, preferably less than 50 nM and most preferably less than 10 μM. Alternatively, affinity may be defined as the ability to inhibit binding of β-galactosidase to M6P receptor in vitro [described by Distler et a/1991 J. Biol. Chem. 266(32) 21687-92], wherein IC₅₀ values of less than 10 μM, preferably less than 1 μM, most preferably less than 0.1 μM and especially preferably less than 0.01 μM. Affinity may also be defined as the ability to inhibit binding of Insulin like Growth Factor-II (IGF-II) to M6P receptor in vitro [Marron P. G. et al 1998 J. Biol. Chem. 273(35) 22358-22366], wherein IC₅₀ values of less than 10 μM, preferably less than 1 μM, most preferably less than 0.1 μM and especially preferably less than 0.01 μM.

The imaging agent of the present invention preferably does not have conjugated to the M6P vector an oligonucleotide, RNA, DNA, peptide nucleic acid, growth factor, vaccine, vitamin, or antibody. Most preferably imaging agent of the present invention has only the imaging moiety conjugated to the M6P vector, i.e. nothing further is conjugated to the M6P vector.

The vector for the M6P receptor preferably comprises at least one of the following:
(i) the 67 amino acid IGF-II sequence, or a fragment or peptide analogue thereof;
(ii) M6P;
(iii) a diphosphorylated glycopeptide; or
(iv) retinoic acid or a derivative thereof.
   IGF-II binds to the M6P receptor with high affinity (Kd 0.3-14 nM at pH 7.4) and is therefore suitable for use as a vector in the present invention. IGF-II is a non-glycosylated single chain of 67 amino acid (aa) residues, as presented in SEQ ID NO. 1. Preferably, a peptide fragment or a peptide analogue of the 67 aa sequence may be used.

The term “peptide fragment” in the context of the present invention is taken to mean a natural or synthetic peptide comprising an isolated section of the 67 aa IGF-II sequence which retains affinity for the M6P receptor. A peptide fragment of the IGF-II sequence may consist of between 5 and 60 aa residues, preferably between 8 and 60 aa residues. Preferred peptide fragments are synthetic peptides.

The term “peptide analogue” in the context of the present invention is taken to mean natural or synthetic peptides comprising all or an isolated section of the 67 aa IGF-II sequence in which one or more aa residues have been substituted with alternative aa residues, and which retains affinity for the M6P receptor. Preferred peptide analogues are synthetic peptides. With the aim of minimizing alteration of the peptide, it is common practice to substitute only a few aa residues and to make only conservative substitutions. The following table outlines substitutions that are regarded as conservative:

| Original aa residue | Exemplary substitutions |
|---|---|
| Ala | Ser |
| Arg | Lys |
| Asn | Gln, His |
| Asp | Glu |
| Cys | Ser, Met |
| Gln | Asn |
| Glu | Asp |
| Gly | Ala, Asn |
| His | Asn, Gln |
| Ile | Leu, Val |
| Leu | Ile, Val |
| Lys | Arg, Gln |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

However, any aa substitution is suitable as long as affinity for the M6P receptor is retained. Furthermore, preferred aa substitutions are those which result in peptide analogues having increased specificity for the M6P receptor over the insulin-like growth factor-I (IGF-I) receptor. For example, the non-conservative substitution of Tyr27 with Leu diminishes affinity for the IGF-I receptor while retaining affinity for the M6P receptor.

The following are examples of peptide fragments and peptide analogues of the 67 aa IGF-II sequence that are most preferred:
(i) a peptide comprising amino acid residues 48-55 (SEQ ID NO 2) or peptide analogues thereof;
(ii) a peptide comprising amino acid residues 8-28 (SEQ ID NO. 3) and 41-61 (SEQ ID NO. 4), or peptide analogues thereof, either joined directly or separated by a linker of Formula -(L³)ₚ-;
(iii) a peptide comprising amino acid residues 8-67 (SEQ ID NO. 5), or peptide analogues thereof; and,
(iv) substitutions of various amino acid residues as follows: Phe26Ser (SEQ ID NO. 6); Phe19Ser (SEQ ID NO. 7); Glu12Lys (SEQ ID NO. 8); Tyr27Leu (SEQ ID NO. 9)
   wherein L³ is as defined for L¹ above and p is 1-30.

An example of a preferred IGF-II fragment of the invention is IGF compound 1:

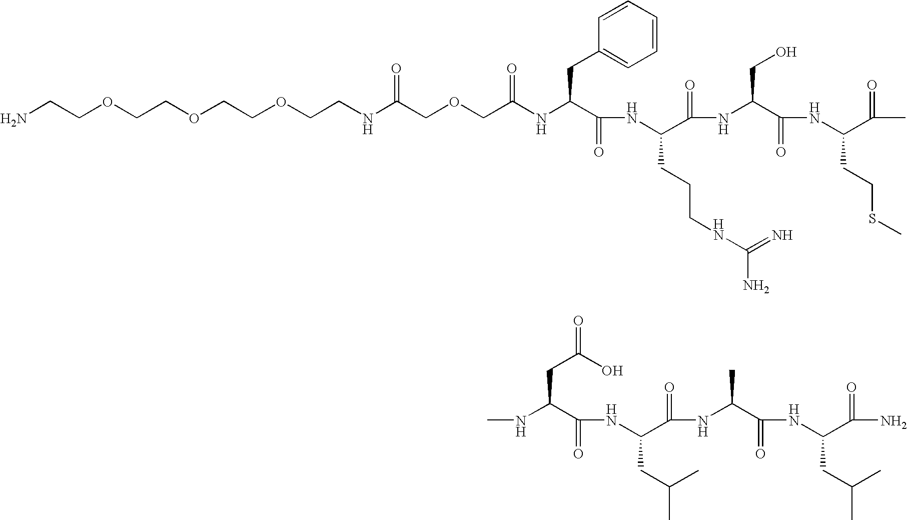

### “IGF Compound 1” (Synthesis Described in Example 1)

These peptides may be obtained by conventional solid phase synthesis. Albericio provides a recent review of methodologies for solid phase peptide synthesis [Curr. Opinion Cell Biol. 2004 8 211-21].

Two or more peptide fragments of IGF-II may be optionally joined by one or more linker groups, -(L³)ₚ-. In the context of linking two peptide fragments of IGF-II, linkers comprising L³ groups that are amino acids and/or PEG are preferred. Where the linker is a PEG linker, it preferably consists of between 1 and 30 ethylene glycol units.

For the IGF-II peptide and fragments or peptide analogues thereof, suitable locations for an imaging moiety include the amino terminus and the carboxy terminus of the peptides. Other suitable locations are the side chains of the amino acids making up the peptide. Preferably, a linker is present connecting IGF-II, or the fragments or peptide analogues thereof, to the imaging moiety. For example, in the case of IGF compound 1 above, an imaging agent is preferably located at the amino group at the end of the linker.

A number of compounds comprising M6P are suitable for use as the vector in the imaging agent of the invention. These include M6P, M6P-decorated human serum albumin (HSA) and M6P-containing oligomannosides, such as those reported by Distler et al [J. Biol. Chem. 1991 266(32) 21687-92] as follows (where Man=mannose):

| | | |
|---|---|---|
| (i) | M6P(α1,2)-Man-O(CH₂)₈CO₂Me | (α1,2 linked dimannoside) |
| (ii) | M6P(α1,3)-Man-O(CH₂)₈CO₂Me | (α1,3 linked dimannoside) |
| (iii) | M6P(α1,6)-Man-O(CH₂)₈CO₂Me | (α1,6 linked dimannoside) |
| (iv) | M6P(α1,2)-Man(α1,2)-Man-O(CH₂)₈CO₂Me | (α1,2 linked trimannoside) |
| (v) | The biantennary oligomannoside: | |

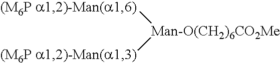

(v) The biantennary oligomannoside:

Compounds (i)-(v) above are referred to herein as “M6P Compound 1” to “M6P Compound 5” respectively. The synthesis of the above oligosaccharide compounds has been reported by Distler et al [J. Biol. Chem. 1991 266(32) 21687-92] and by Srivastava et al [J. Org. Chem. 1987 52 2869-75].

Where a compound comprising M6P is the vector, an imaging moiety is suitably conjugated via a substituent of said vector that is not involved in binding to the M6P receptor. Preferably, the compound comprising M6P is one of M6P Compounds 1-6, and the imaging moiety is conjugated via the carboxymethyl group of M6P Compounds 1-5, or via the lysine residue amine of M6P Compound 6.

In an alternative preferred embodiment, the compound comprising M6P can be one of the above-described compounds but wherein one or more of the phosphate groups is replaced with a phosphonate group.

Other compounds suitable for use as the vector in the imaging agent of the invention are diphosphorylated glycopeptides. WO 95/014036 discloses such compounds which bind to M6P receptor and are useful in the treatment of inflammatory and other diseases and their synthesis is described by Christensen et al (J. Chem. Soc. Perkin Trans. 1994 1299-1310). Any of these compounds are suitable vectors for the present invention. They may be represented by Formula I:

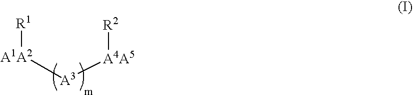

wherein
R¹ and R² are independently selected from
(i) a natural L- or D-monosaccharide chosen from: glucose, mannose, galactose, fucose, rhammanose, N-acetylglucosamine, N-acetylgalactosaminyl, fructose and N-acetylneuraminic acid, or phosphorylated or sulphated versions thereof; or,
(ii) an oligosaccharide composed of monosaccharides selected from (i);
   A¹ and A⁵ are independently selected from the group consisting of —H, —OH, —NH₂, -acetyl, D- or L-amino acids, peptides, glycopeptides, peptidomimetics and oligonucleotides,
   A² and A⁴ are independently selected from the group of D- or L-hydroxy amino acids, e.g. Ser, Thr, Hyl, Hyp, Tyr or D- or L-carboxamido amino acids, e.g. Asn and Gln, and
A³ is selected from the group of genetically encoded or non-encoded amino acids in their D- or L-form or peptidomimetics or nucleotides, and wherein m is an integer between 1 and 30 and wherein any residue in the linear sequence A¹-A⁵ may be covalently linked to form a cyclic compound.

Preferably, R¹ and R² are M6P groups, A¹ is an acetyl group and A⁵ is —NH₂, A² and A⁴ are Thr, and A³ is a chain of between 1 and 5 amino acid residues.

Most preferred diphosphorylated glycopeptides of the present invention are:
(i) Ac-Thr[α-D-M6P-(1,2)-α-D-mannose]-Lys(aminobenzamide)-Thr[α-D-M6P-(1,2)-αD-mannose]-NH₂ (“Glycopeptide Compound 1”)
(ii) Ac-Thr[α-D-M6P]-Gly-Lys-Gly-Thr[α-D-M6P]—NH₂ (“Glycopeptide Compound 2”)

Where a diphosphorylated glycopeptide is the vector with affinity for the M6P receptor, the imaging moiety is preferably conjugated via one of the amino acids present in the group (A³)ₘ.

In an alternative preferred embodiment, the diphosphorylated glycopeptide can be one of the above-described compounds but wherein one or more of the phosphate groups is replaced with a phosphonate group.

Retinoic acid binds to a site on M6P receptor distinct from the IGF-II binding site and with high affinity (Kd 2.4 nM). It and its derivatives are therefore suitable as vectors in the imaging agent of the present invention. The structure of retinoic acid is illustrated below:

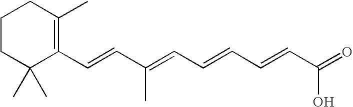

Derivatives of retinoic acid suitable for the invention include retinoic acid modified by the addition of the group of Formula -(L⁴)_{q}-R″, wherein L⁴, q and R″ are as defined above for L¹, n and R. R″ is preferably amino, carboxy and hydroxyl and L⁴ is preferably an amino acid or PEG. Where the linker is a PEG linker, it preferably consists of between 1 and 20 ethylene glycol units, most preferably between 1 and 15 ethylene glycol units and especially preferably between 1 and 12 ethylene glycol units.

Preferred derivatives of retinoic acid of this type include a PEG linker plus one or more amino acid residues. It is envisaged that the PEG linker may include one or more charged amino acid groups as a means to adjust the biodistribution and/or excretion of the resultant imaging agent.

Where the vector for the M6P receptor is retinoic acid or an analogue thereof, the imaging moiety is preferably conjugated via the carboxy group of retinoic acid or via any a reactive group present in a retinoic acid analogue, e.g. an amino or carboxy group in Retinoic acid Compound 1 above.

It is furthermore envisaged that the vector may be a multivalent targeting vector combining two or more of the above-described vectors. Imaging agents comprising such vectors are anticipated to exhibit an increased affinity for M6P receptor due to the fact that M6P receptor contains independent binding sites for each of the vectors. Examples of such multivalent vectors are illustrated below:

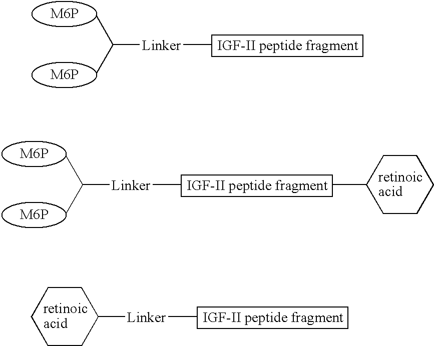

For multivalent vectors with two M6P groups, these are preferably joined together with a branched PEG linker group, as defined previously. The linker illustrated in the above diagrams is of Formula -(L⁵)ᵣ- wherein L⁵ is as defined previously for L¹ and r is 10 to 50. Ideally, the linker acts to space apart the individual vector moieties such that they are positioned to bind optimally with their respective binding sites on the M6P receptor. Linkers comprising amino acids and/or PEG are preferred.

Following administration of the imaging agent, the “imaging moiety” may be detected either external to the human body or via use of detectors designed for use in vivo, such as intravascular radiation or optical detectors such as endoscopes, or radiation detectors designed for intra-operative use.

The imaging moiety is preferably chosen from:
(i) a radioactive metal ion;
(ii) a paramagnetic metal ion;
(iii) a gamma-emitting radioactive halogen;
(iv) a positron-emitting radioactive non-metal;
(v) a hyperpolarised NMR-active nucleus;
(vi) a reporter suitable for in vivo optical imaging;
(vii) a β-emitter suitable for intravascular detection.

When the imaging moiety is a radioactive metal ion, i.e. a radiometal, suitable radiometals can be either positron emitters such as ⁶⁴Cu, ⁴⁸V, ⁵²Fe, ⁵⁵Co, ^{94m}Tc or ⁶⁸Ga; γ-emitters such as ^{99m}Tc, ¹¹¹In, ^{113m}In, or ⁶⁷Ga. Preferred radiometals are ^{99m}Tc, ⁶⁴Cu, ⁶⁸Ga and ¹¹¹In. Most preferred radiometals are γ-emitters, especially ^{99m}Tc.

When the imaging moiety is a paramagnetic metal ion, suitable such metal ions include: Gd(III), Mn(II), Cu(II), Cr(III), Fe(III), Co(II), Er(II), Ni(II), Eu(III) or Dy(III). Preferred paramagnetic metal ions are Gd(III), Mn(II) and Fe(III), with Gd(III) being especially preferred.

When the imaging moiety is a gamma-emitting radioactive halogen, the radiohalogen is suitably chosen from ¹²³I, ¹³¹I or ⁷⁷Br. ¹²⁵I is specifically excluded as it is not suitable for use as an imaging moiety for diagnostic imaging. A preferred gamma-emitting radioactive halogen is ¹²³I.

When the imaging moiety is a positron-emitting radioactive non-metal, suitable such positron emitters include: ¹¹C, ¹³N, ¹⁵O, ¹⁷F, ¹⁸F, ⁷⁵Br, ⁷⁶Br or ¹²⁴I. Preferred positron-emitting radioactive non-metals are ¹¹C, ¹³N, ¹⁸F and ¹²⁴I, especially ¹¹C and ¹⁸F, most especially ¹⁸F.

When the imaging moiety is a hyperpolarised NMR-active nucleus, such NMR-active nuclei have a non-zero nuclear spin, and include ¹³C, ¹⁵N, ¹⁹F, ²⁹Si and ³¹P. Of these, ¹³C is preferred. By the term “hyperpolarised” is meant enhancement of the degree of polarisation of the NMR-active nucleus over its' equilibrium polarisation. The natural abundance of ¹³C (relative to ¹²C) is about 1%, and suitable ¹³C-labelled compounds are suitably enriched to an abundance of at least 5%, preferably at least 50%, most preferably at least 90% before being hyperpolarised. At least one carbon atom of the imaging agent of the invention is suitably enriched with ¹³C, which is subsequently hyperpolarised.

When the imaging moiety is a reporter suitable for in vivo optical imaging, the reporter is any moiety capable of detection either directly or indirectly in an optical imaging procedure. The reporter might be a light scatterer (e.g. a coloured or uncoloured particle), a light absorber or a light emitter. More preferably the reporter is a dye such as a chromophore or a fluorescent compound. The dye can be any dye that interacts with light in the electromagnetic spectrum with wavelengths from the ultraviolet light to the near infrared. Most preferably the reporter has fluorescent properties.

Preferred organic chromophoric and fluorophoric reporters include groups having an extensive delocalized electron system, e.g. cyanines, merocyanines, indocyanines, phthalocyanines, naphthalocyanines, triphenylmethines, porphyrins, pyrilium dyes, thiapyrilium dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, bis(dithiolene) complexes, bis(benzene-dithiolate) complexes, iodoaniline dyes, bis(S,O-dithiolene) complexes. Fluorescent proteins, such as green fluorescent protein (GFP) and modifications of GFP that have different absorption/emission properties are also useful. Complexes of certain rare earth metals (e.g., europium, samarium, terbium or dysprosium) are used in certain contexts, as are fluorescent nanocrystals (quantum dots).

Particular examples of chromophores which may be used include: fluorescein, sulforhodamine 101 (Texas Red), rhodamine B, rhodamine 6G, rhodamine 19, indocyanine green, Cy2, Cy3, Cy3.5, Cy5, Cy5.5, Cy7, Marina Blue, Pacific Blue, Oregon Green 88, Oregon Green 514, tetramethylrhodamine, and Alexa Fluor 350, Alexa Fluor 430, Alexa Fluor 532, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, and Alexa Fluor 750.

Particularly preferred are dyes which have absorption maxima in the visible or near infrared (NIR) region, between 400 nm and 3 μm, particularly between 600 and 1300 nm. Optical imaging modalities and measurement techniques include, but not limited to: luminescence imaging; endoscopy; fluorescence endoscopy; optical coherence tomography; transmittance imaging; time resolved transmittance imaging; confocal imaging; nonlinear microscopy; photoacoustic imaging; acousto-optical imaging; spectroscopy; reflectance spectroscopy; interferometry; coherence interferometry; diffuse optical tomography and fluorescence mediated diffuse optical tomography (continuous wave, time domain and frequency domain systems), and measurement of light scattering, absorption, polarisation, luminescence, fluorescence lifetime, quantum yield, and quenching.

When the imaging moiety is a β-emitter suitable for intravascular detection, suitable such β-emitters include the radiometals ⁶⁷Cu, ⁸⁹Sr, ⁹⁰Y, ¹⁵³Sm, ¹⁸⁶Re, ¹⁸⁸Re or ¹⁹²Ir, and the non-metals ³²P, ³³P, ³⁸S, ³⁸Cl, ³⁹Cl, ⁸²Br and ⁸³Br.

Preferred imaging moieties are those which can be detected externally in a non-invasive manner following administration in vivo. Most preferred imaging moieties are radioactive, especially radioactive metal ions, gamma-emitting radioactive halogens and positron-emitting radioactive non-metals, particularly those suitable for imaging using SPECT or PET. The various means by which these imaging moieties can be incorporated into each of the vector types are outlined below in the description of a further aspect of the invention.

Preferred imaging agents of the invention do not undergo facile metabolism in vivo, and hence most preferably exhibit a half-life in vivo of 60 to 240 minutes in humans. The imaging agent is preferably excreted via the kidney (i.e. exhibits urinary excretion). The imaging agent preferably exhibits a signal-to-background ratio at diseased foci of at least 1.5, most preferably at least 5, with at least 10 being especially preferred. Where the imaging agent comprises a radioisotope, clearance of one half of the peak level of imaging agent which is either non-specifically bound or free in vivo, preferably occurs over a time period less than or equal to the radioactive decay half-life of the radioisotope of the imaging moiety.

Furthermore, the molecular weight of the imaging agent is suitably up to 5000 Daltons. Preferably, the molecular weight is in the range 150 to 3000 Daltons, most preferably 200 to 1500 Daltons, with 300 to 800 Daltons being especially preferred.

Examples of imaging agents of the invention are illustrated below:

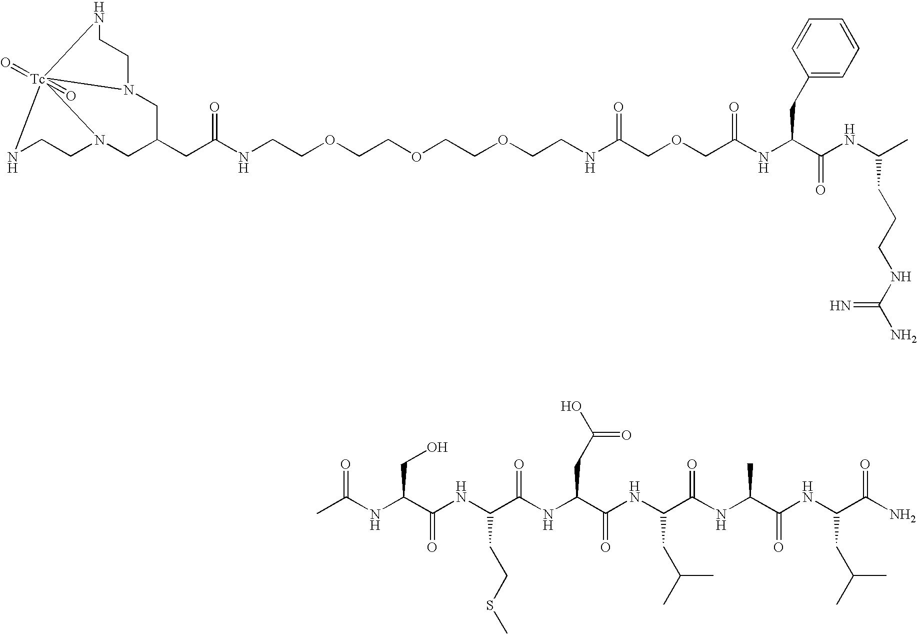

### IGF Imaging Agent 1

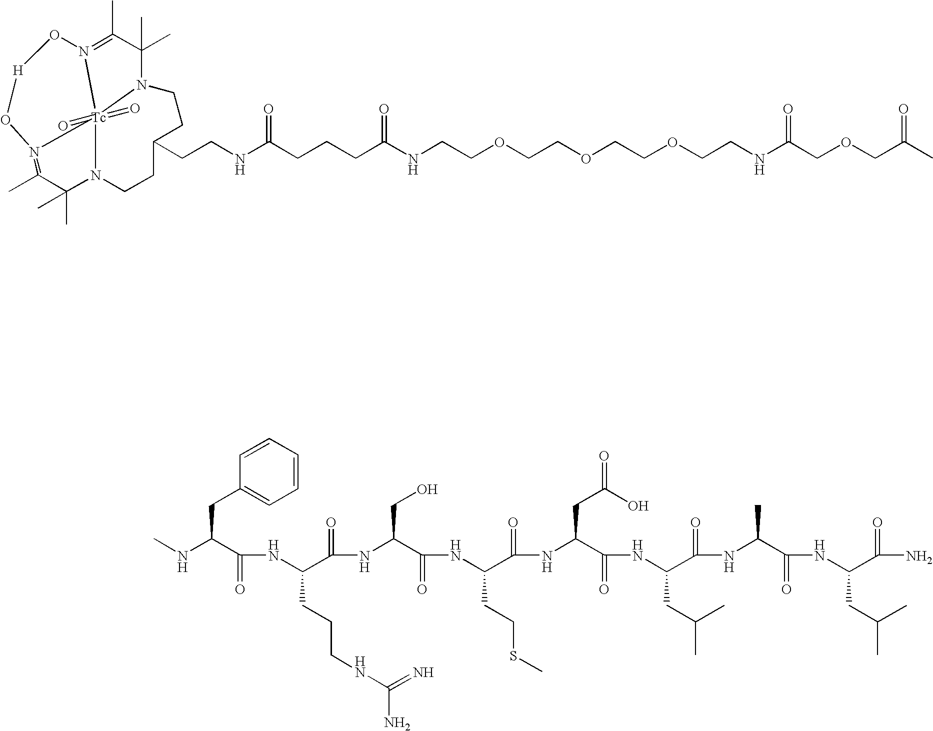

### IGF Imaging Agent 2

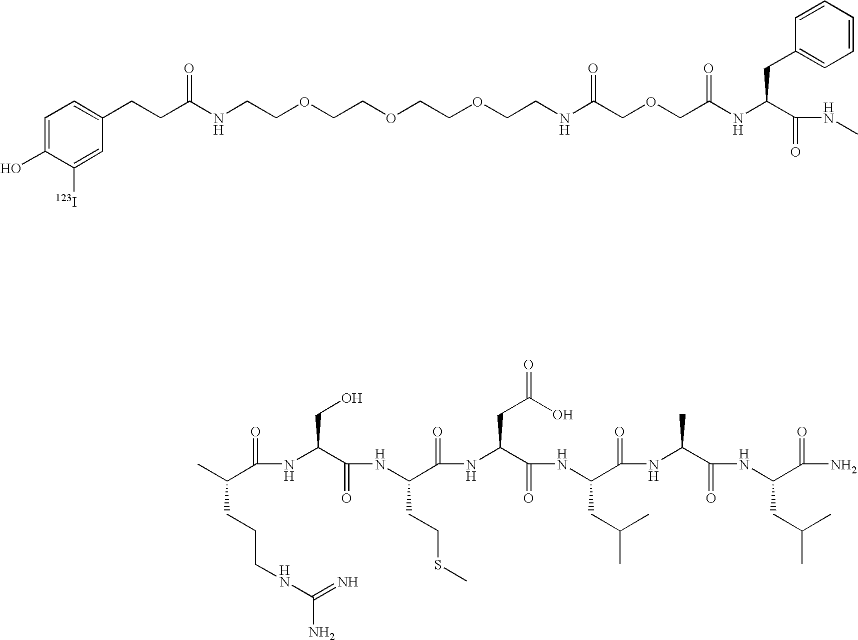

F imaging Agent 3

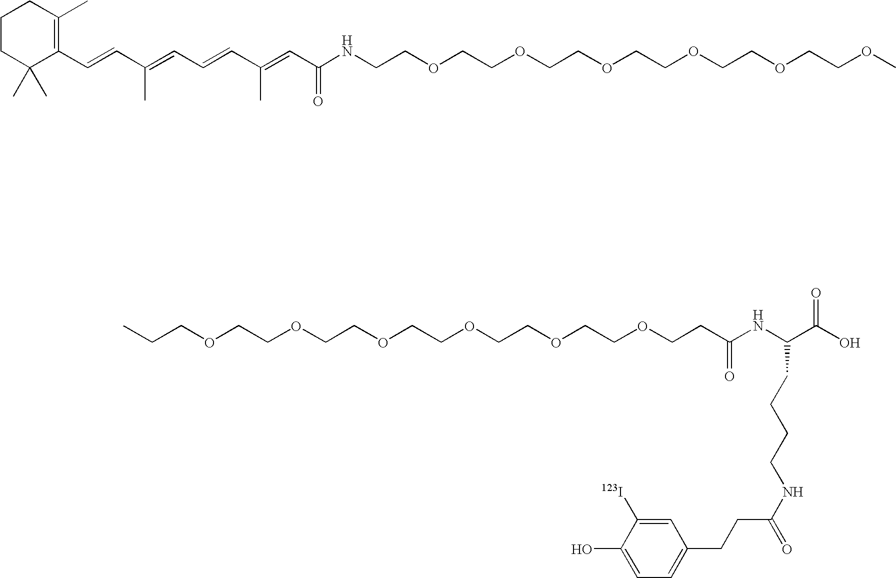

### REtinoic Acid Imaging Agent 1

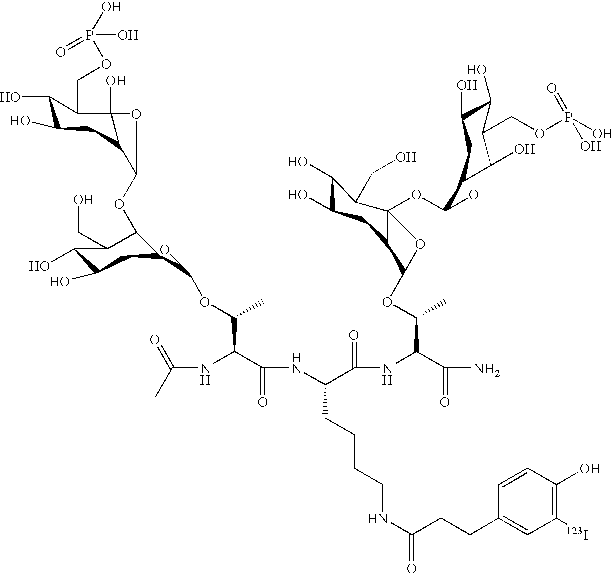

### Glycopeptide Imaging Agent 1

Synthetic routes for obtaining the above compounds are described in Examples 4, 7, 8 and 10, respectively.

The imaging agents of this aspect of the invention are prepared from precursor compounds, which embody a further aspect of the invention and are described in more detail below.

In a further aspect, the present invention provides a method for the preparation of the imaging agent of the invention comprising reaction of a precursor with a suitable source of an imaging moiety wherein said precursor comprises:

(i) a vector with affinity for M6P receptor; and

(ii) a chemical group capable of reacting with a source of the imaging moiety so that the imaging moiety becomes attached to the compound to result in said imaging agent;

wherein said chemical group is either an integral part of said vector or is conjugated to said vector.

A “precursor” comprises a derivative of the vector, designed so that chemical reaction with a convenient chemical form of the imaging moiety occurs site-specifically; can be conducted in the minimum number of steps (ideally a single step); and without the need for significant purification (ideally no further purification), to give the desired imaging agent. Such precursors are synthetic and con conveniently be obtained in good chemical purity. The “precursor” may optionally comprise a protecting group for certain functional groups of the vector with affinity for M6P receptor.

By the term “protecting group” is meant a group which inhibits or suppresses undesirable chemical reactions, but which is designed to be sufficiently reactive that it may be cleaved from the functional group in question under mild enough conditions that do not modify the rest of the molecule. After deprotection the desired product is obtained. Protecting groups are well known to those skilled in the art and are suitably chosen from, for amine groups: Boc (where Boc is tert-butyloxycarbonyl), Fmoc (where Fmoc is fluorenylmethoxycarbonyl), trifluoroacetyl, allyloxycarbonyl, Dde [i.e. 1-(4,4-dimethyl-2,6-dioxocyclohexylidene)ethyl] or Npys (i.e. 3-nitro-2-pyridine sulfenyl); and for carboxyl groups: methyl ester, tert-butyl ester or benzyl ester. For hydroxyl groups, suitable protecting groups are: methyl, ethyl or tert-butyl; alkoxymethyl or alkoxyethyl; benzyl; acetyl; benzoyl; trityl (Trt) or trialkylsilyl such as tetrabutyldimethylsilyl. For thiol groups, suitable protecting groups are: trityl and 4-methoxybenzyl. The use of further protecting groups are described in ‘Protective Groups in Organic Synthesis’, Theorodora W. Greene and Peter G. M. Wuts, (Third Edition, John Wiley & Sons, 1999).

Preferably, said chemical group capable of reacting with a source of an imaging moiety comprises:
(i) a chelator capable of complexing a metallic imaging moiety;
(ii) an organometallic derivative such as a trialkylstannane or a trialkylsilane;
(iii) a derivative containing an alkyl halide, alkyl tosylate or alkyl mesylate for nucleophilic substitution;
(iv) a derivative containing an aromatic ring activated towards nucleophilic or electrophilic substitution;
(v) a derivative containing a functional group which undergoes facile alkylation; or,
(vi) a derivative which alkylates thiol-containing compounds to give a thioether-containing product.

When the imaging moiety comprises a metal ion, the precursor comprises a chemical group capable of complexing the metal ion to form a metal complex. By the term “metal complex” is meant a coordination complex of the metal ion with one or more ligands. It is strongly preferred that the metal complex is “resistant to transchelation”, i.e. does not readily undergo ligand exchange with other potentially competing ligands for the metal coordination sites. Potentially competing ligands include the vector for M6P receptor itself plus other excipients in the preparation in vitro (e.g. radioprotectants or antimicrobial preservatives used in the preparation), or endogenous compounds in vivo (e.g. glutathione, transferrin or plasma proteins).

Suitable ligands for use in the present invention which form metal complexes resistant to transchelation include: chelating agents, where 2-6, preferably 2-4, metal donor atoms are arranged such that 5- or 6-membered chelate rings result (by having a non-coordinating backbone of either carbon atoms or non-coordinating heteroatoms linking the metal donor atoms); or monodentate ligands which comprise donor atoms which bind strongly to the metal ion, such as isonitriles, phosphines or diazenides. Examples of donor atom types which bind well to metals as part of chelating agents are: amines, thiols, amides, oximes, and phosphines. Phosphines form such strong metal complexes that even monodentate or bidentate phosphines form suitable metal complexes. The linear geometry of isonitriles and diazenides is such that they do not lend themselves readily to incorporation into chelating agents, and are hence typically used as monodentate ligands. Examples of suitable isonitriles include simple alkyl isonitriles such as tert-butylisonitrile, and ether-substituted isonitriles such as mibi (i.e. 1-isocyano-2-methoxy-2-methylpropane). Examples of suitable phosphines include Tetrofosmin, and monodentate phosphines such as tris(3-methoxypropyl)phosphine. Examples of suitable diazenides include the HYNIC series of ligands i.e. hydrazine-substituted pyridines or nicotinamides.

Examples of suitable chelating agents for technetium which form metal complexes resistant to transchelation include, but are not limited to:

(i) diaminedioximes;
(ii) N₃S ligands having a thioltriamide donor set such as MAG₃ (mercaptoacetyltriglycine) and related ligands; or having a diamidepyridinethiol donor set such as Pica;
(iii) N₂S₂ ligands having a diaminedithiol donor set such as BAT or ECD (i.e. ethylcysteinate dimer), or an amideaminedithiol donor set such as MAMA;
(iv) N₄ ligands which are open chain or macrocyclic ligands having a tetramine, amidetriamine or diamidediamine donor set, such as cyclam, monoxocyclam or dioxocyclam; or,
(v) N₂O₂ ligands having a diaminediphenol donor set.

Preferred chelating agents of the invention for technetium are diaminedioximes and tetraamines, the preferred versions of which are now described in more detail.

Preferred diaminedioximes are of Formula (X):

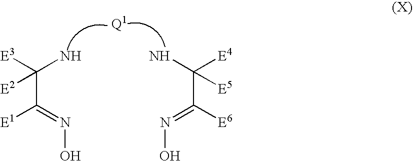

where E¹-E⁶ are each independently an R* group;
each R* is H or Cl₁₀ alkyl, C₃₋₁₀ alkylaryl, C₂₋₁₀ alkoxyalkyl, C₁₋₁₀ hydroxyalkyl, C₁₋₁₀fluoroalkyl, C₂₋₁₀ carboxyalkyl or C₁₋₁₀ aminoalkyl, or two or more R* groups together with the atoms to which they are attached form a carbocyclic, heterocyclic, saturated or unsaturated ring, and wherein one or more of the R* groups is conjugated to the vector;
and Q¹ is a bridging group of formula -(J¹)_{f}-;
where f is 3, 4 or 5 and each J¹ is independently —O—, —NR*— or —C(R*)₂— provided that -(J¹)_{f}- contains a maximum of one J¹ group which is —O— or —NR*—.

Preferred Q¹ groups are as follows:

Q¹=-(CH₂)(CHR*)(CH₂)— i.e. propyleneamine oxime or PnAO derivatives;
Q=-(CH₂)₂(CHR*)(CH₂)₂— i.e. pentyleneamine oxime or PentAO derivatives;

### Q¹=-(CH₂)₂NR*(CH₂)₂—.

E¹ to E⁶ are preferably chosen from: C₁₋₃ alkyl, alkylaryl alkoxyalkyl, hydroxyalkyl, fluoroalkyl, carboxyalkyl or aminoalkyl. Most preferably, each E¹ to E⁶ group is CH₃.

The vector for M6P receptor is preferably conjugated at either the E¹ or E⁶R* group, or an R* group of the Q¹ moiety. Most preferably, it is conjugated to an R* group of the Q¹ moiety. When it is conjugated to an R* group of the Q¹ moiety, the R* group is preferably at the bridgehead position. In that case, Q¹ is preferably —(CH₂)(CHR*)(CH₂)—, —(CH₂)₂(CHR*)(CH₂)₂— or —(CH₂)₂NR*(CH₂)₂—, most preferably —(CH₂)₂(CHR*)(CH₂)₂—. An especially preferred bifunctional diaminedioxime chelator has the Formula (Xa):

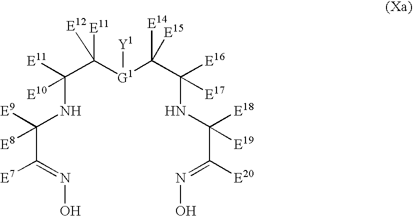

where:
E⁷-E²⁰ are each independently an R* group as defined above;

### G¹ is N or CR*;

Y¹ is -(L⁶)ₛ-vector, wherein L⁶ and s are as defined for L¹ and n above, and ‘vector’ represents a vector with affinity for the M6P receptor as previously defined. Where there is a linker group -(L⁶)ₛ-, there is no other linker group joining the chelate and the vector.

A preferred chelator of Formula (Xa) is of Formula (Xb):

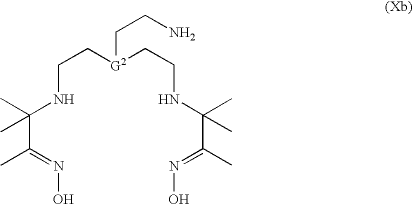

where G² is defined as for G¹ above, and is preferably CH (=“chelate X” for which the synthesis is described in Example 5);
such that the vector for M6P receptor is conjugated via the bridgehead —CH₂CH₂NH₂ group.

Preferred tetraamine chelators of the invention are of Formula Z:

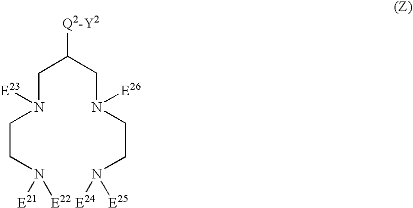

wherein:
Q² is a bridging group of formula -(J²)_{g}-;
wherein g is 1-8 and each J² is independently —O—, —NR*— or —C(R*)₂—, preferably —C(R*)₂— and most preferably —CH₂—, wherein R* is as defined previously.
Y² is the group -(L⁷)ₜ-vector, wherein L⁷ and t are as previously defined for L¹ and n. Where there is a linker -(L⁷)ₜ-, no other linker joins the chelate to the vector. Preferably for these tetraamine chelates, the linker does not contain aryl rings. This helps to minimize the lipophilicity of the complex.
E²¹ to E²⁶ are an R* group as previously defined.

A most preferred tetraamine chelate of the present invention is of Formula Za:

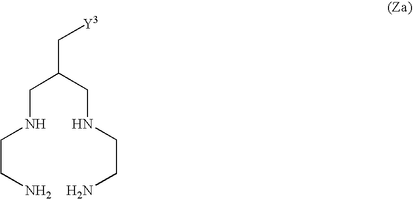

wherein Y³ is as defined above for Y².

An especially preferred tetraamine chelate of the present invention is of Formula Za wherein

Y³ is —CO-vector (“chelate Z” synthesis of chelate without vector attached described in Example 2).

The above described ligands are particularly suitable for complexing technetium e.g. ^{94m}Tc or ^{99m}Tc, and are described more fully by Jurisson et al [Chem. Rev., 99, 2205-2218 (1999)]. The ligands are also useful for other metals, such as copper (⁶⁴Cu or ⁶⁷Cu), vanadium (e.g. ⁴⁸V), iron (e.g. ⁵²Fe), or cobalt (e.g. ⁵⁵Co). Other suitable ligands are described in Sandoz WO 91/01144, which includes ligands which are particularly suitable for indium, yttrium and gadolinium, especially macrocyclic aminocarboxylate and aminophosphonic acid ligands. Examples of suitable chelating agents of this type include 1,4,7,10-tetraazacyclododecane-1,4,7,10-tetraacetic acid (DOTA) and diethylenetriaminepentaacetic acid (DTPA). Ligands which form non-ionic (i.e. neutral) metal complexes of gadolinium are known and are described in U.S. Pat. No. 4,885,363. When the radiometal ion is technetium, the ligand is preferably a chelating agent which is tetradentate. Preferred chelating agents for technetium are the diaminedioximes, or those having an N₂S₂ or N₃S donor set as described above.

It is envisaged that the role of the linker group [defined above as either -(L⁶)ₛ- or -(L⁷)ₜ-] is to distance the relatively bulky metal complex, which results upon metal coordination, from the active site of the vector for M6P receptor so that e.g. receptor binding is not impaired. This can be achieved by a combination of flexibility (e.g. simple alkyl chains), so that the bulky group has the freedom to position itself away from the active site and/or rigidity such as a cycloalkyl or aryl spacer which orientates the metal complex away from the active site. The nature of the linker group can also be used to modify the biodistribution and excretion properties of the resulting metal complex of the conjugate. Thus, e.g. the introduction of ether groups in the linker will help to minimise plasma protein binding, or the use of polymeric linker groups such as polyalkyleneglycol, especially PEG (polyethyleneglycol) can help to prolong the lifetime of the agent in the blood in vivo.

Preferred linker groups -(L⁶)ₛ- or -(L⁷)ₜ- have a backbone chain which contains 2 to 10 atoms, most preferably 2 to 5 atoms, with 2 or 3 atoms being especially preferred. A minimum linker group backbone chain of 2 atoms confers the advantage that the chelator is well-separated from the biological targeting moiety so that any interaction is minimised. Furthermore, the vector is unlikely to compete effectively with the coordination of the chelator to the metal ion. In this way, both the biological targeting characteristics of the vector, and the metal complexing capability of the chelator is maintained. It is strongly preferred that the vector for M6P receptor is bound to the chelator in such a way that the linkage does not undergo facile metabolism in blood. That is because such metabolism would result in the imaging metal complex being cleaved off before the labelled vector for M6P receptor reaches the desired in vivo target site. The vector for M6P receptor is therefore preferably covalently bound to the metal complexes of the present invention via linker groups which are not readily metabolised. Suitable such linkages are carbon-carbon bonds, amide bonds, urea or thiourea linkages, or ether bonds.

Non-peptide linker groups such as alkylene groups or arylene groups have the advantage that there are no significant hydrogen bonding interactions with the conjugated vector for M6P receptor so that the linker does not wrap round onto the vector. Preferred alkylene spacer groups are —(CH₂)ᵤ— where u is an integer of value 2 to 5. Preferably q is 2 or 3.

Preferred arylene spacers are of formula:

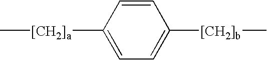

where: a and b are each independently 0, 1 or 2.
Preferred Y¹-Y³ groups are thus —CH₂CH₂-(L⁸)ᵥ-,—where v is an integer of value 0 to 3.

When the vector is a peptide, Y¹-Y³ is preferably —CH₂CH₂-(L⁹)_{w}- where L⁹ is —CO— or —NR′″— and w is 0 to 3, wherein R′″ is as defined for R above. When either G¹ or G² is N and -(L⁹)_{w-} is —NH—, this grouping has the additional advantage that it stems from the symmetrical intermediate N(CH₂CH₂NH₂)₃, which is commercially available.

When the imaging metal is technetium, the usual technetium starting material is pertechnetate, i.e. TcO₄— which is technetium in the Tc(VII) oxidation state. Pertechnetate itself does not readily form metal complexes, hence the preparation of technetium complexes usually requires the addition of a suitable reducing agent such as stannous ion to facilitate complexation by reducing the oxidation state of the technetium to the lower oxidation states, usually Tc(I) to Tc(V). The solvent may be organic or aqueous, or mixtures thereof. When the solvent comprises an organic solvent, the organic solvent is preferably a biocompatible solvent, such as ethanol or DMSO. Preferably the solvent is aqueous, and is most preferably isotonic saline.

Where the imaging moiety is radioiodine, preferred precursors are those which comprise a derivative which either undergoes electrophilic or nucleophilic iodination or undergoes condensation with a labelled aldehyde or ketone. Examples of the first category are:
(a) organometallic derivatives such as a trialkylstannane (eg. trimethylstannyl or tributylstannyl), or a trialkylsilane (eg. trimethylsilyl) or an organoboron compound (eg. boronate esters or organotrifluoroborates);
(b) a non-radioactive alkyl bromide for halogen exchange or alkyl tosylate, mesylate or triflate for nucleophilic iodination;
(c) aromatic rings activated towards electrophilic iodination (eg. phenols) and aromatic rings activated towards nucleophilic iodination (eg. aryl iodonium salt aryl diazonium, aryl trialkylammonium salts or nitroaryl derivatives).

The precursor preferably comprises: a non-radioactive halogen atom such as an aryl iodide or bromide (to permit radioiodine exchange); an activated precursor aryl ring (e.g. a phenol group); an organometallic precursor compound (e.g. trialkyltin, trialkylsilyl or organoboron compound); or an organic precursor such as triazenes or a good leaving group for nucleophilic substitution such as an iodonium salt. Preferably for radioiodination, the precursor comprises an organometallic precursor compound, most preferably trialkyltin.

Precursors and methods of introducing radioiodine into organic molecules are described by Bolton [J. Lab. Comp. Radiopharm., 45, 485-528 (2002)]. Precursors and methods of introducing radioiodine into proteins are described by Wilbur [Bioconj. Chem., 3(6), 433-470 (1992)]. Suitable boronate ester organoboron compounds and their preparation are described by Kabalaka et al [Nucl. Med. Biol., 29, 841-843 (2002) and 30, 369-373 (2003)]. Suitable organotrifluoroborates and their preparation are described by Kabalaka et al [Nucl. Med. Biol., 31, 935-938 (2004)].

Examples of aryl groups to which radioactive iodine can be attached are given below:

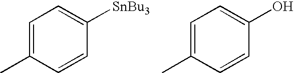

Both contain substituents which permit facile radioiodine substitution onto the aromatic ring. Alternative substituents containing radioactive iodine can be synthesised by direct iodination via radiohalogen exchange, e.g.

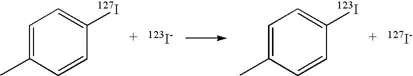

The radioiodine atom is preferably attached via a direct covalent bond to an aromatic ring such as a benzene ring, or a vinyl group since it is known that iodine atoms bound to saturated aliphatic systems are prone to in vivo metabolism and hence loss of the radioiodine.

When the imaging moiety is a radioactive isotope of fluorine the radiofluorine atom may form part of a fluoroalkyl or fluoroalkoxy group, since alkyl fluorides are resistant to in vivo metabolism. Alternatively, the radiofluorine atom may be attached via a direct covalent bond to an aromatic ring such as a benzene ring. Radiohalogenation may be carried out via direct labelling using the reaction of ¹⁸F-fluoride with a suitable chemical group in the precursor having a good leaving group, such as an alkyl bromide, alkyl mesylate or alkyl tosylate. ¹⁸F can also be introduced by alkylation of N-haloacetyl groups with a ¹⁸F(CH₂)₃OH reactant, to give —NH(CO)CH₂—O—(CH₂)₃¹⁸F derivatives. For aryl systems, ¹⁸F-fluoride nucleophilic displacement from an aryl diazonium salt, aryl nitro compound or an aryl quaternary ammonium salt are suitable routes to aryl-¹⁸F derivatives.

A further approach for radiofluorination as described in WO 03/080544, is to react a precursor compound comprising one of the following substituents:

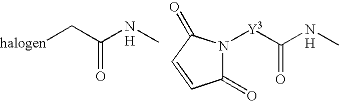

with a compound of Formula V:

¹⁸F—Y⁴—SH (V)

wherein Y³ is a linker of formula -(L¹⁰)ₓ- wherein L¹⁰ is as previously defined for L¹, x is 1-10 and optionally includes 1-6 heteroatoms; and,
Y⁴ is a linker of formula -(L¹¹)_{y}- wherein L¹¹ is as previously defined for L¹, y is 1-30 and optionally includes 1 to 10 heteroatoms;
to give radiofluorinated imaging agents of formula (Va) or (Vb) respectively:

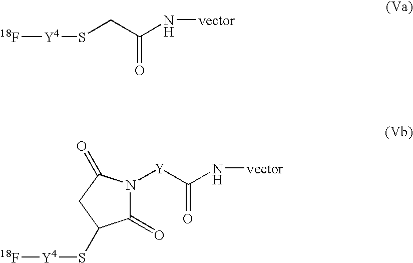

wherein Y⁴ is as defined above, and ‘vector’ is a vector with affinity for the M6P receptor, as defined above in relation to the imaging agent of the invention.

Further details of synthetic routes to ¹⁸F-labelled derivatives are described by Bolton, J. Lab. Comp. Radiopharm., 45, 485-528 (2002).

A ¹⁸F-labelled compound of the invention may be obtained by formation of ¹⁸F fluorodialkylamines and subsequent amide formation when the ¹⁸F fluorodialkylamine is reacted with a precursor containing, e.g. chlorine, P(O)Ph₃ or an activated ester.

Where the vector is IGF-II or a fragment or analogue thereof, the imaging moiety may be incorporated via either their amino- or carboxy terminus. Where a linker is present, the imaging moiety may be incorporated via any reactive group therein. For example, a linker may be added to the amino terminus of IGF-II aa 48-55 followed by addition of a chemical group suitable for the incorporation of an imaging moiety to produce precursor compounds such as the following:

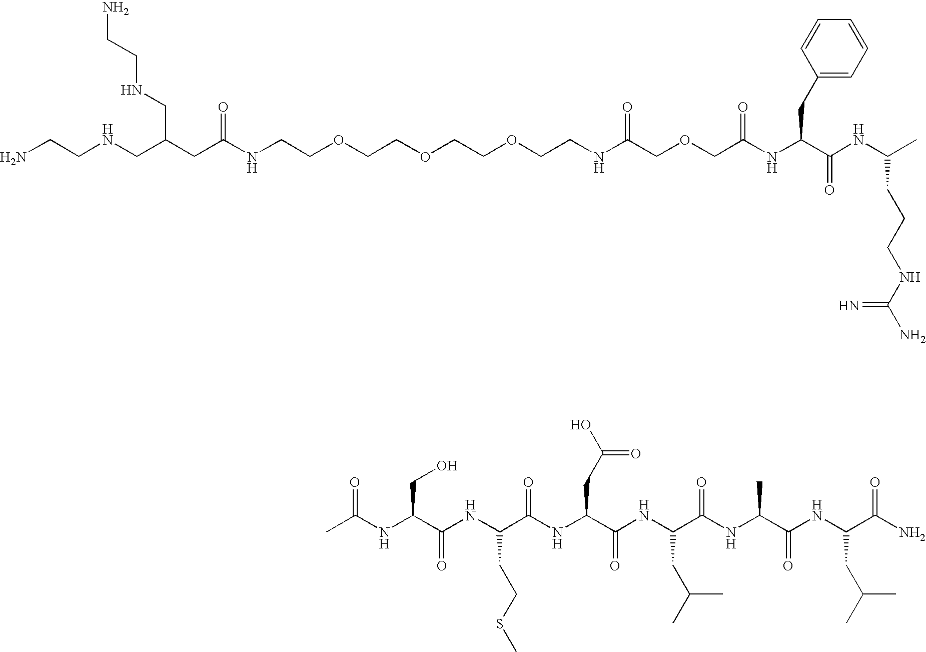

### “IGF Precursor 1” (Synthesis Described in Example 3)

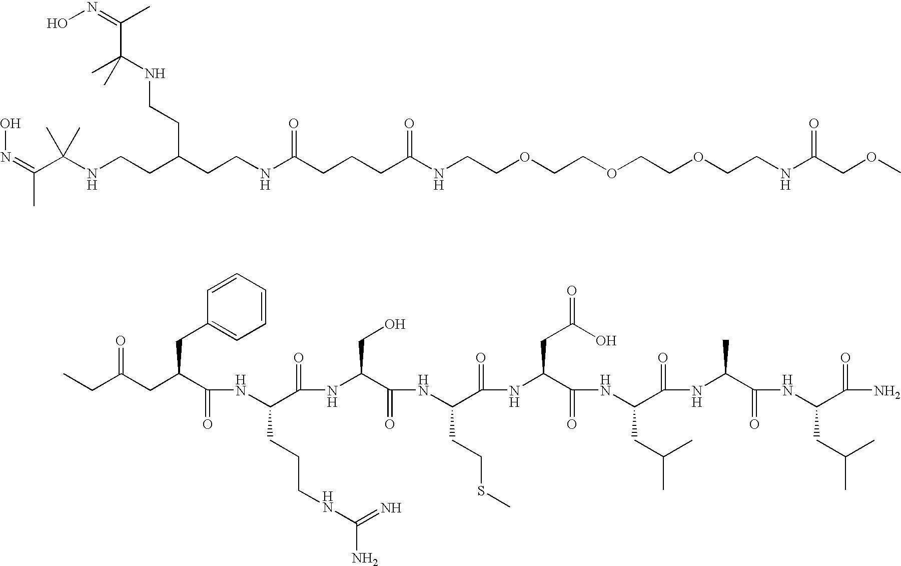

### “IGF Precursor 2” (Synthesis Described in Example 6)

Where the vector comprises M6P, the imaging moiety may be incorporated by derivatisation of the carboxymethyl group of the preferred compounds described above to produce precursor compounds such as:

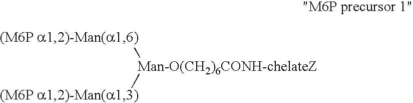

Where the vector is a diphosphorylated glycopeptide, the imaging moiety may for example be incorporated via one of the amino acid residues present in the peptide linking the M6P units to form precursor compounds such as:
(iii) Ac-Thr[α-D-M6P-(1,2)-α-D-mannose]-Lys(chelate)-Thr[α-D-M6P-(1,2)-α-D-mannose]-NH₂ (“glycopeptide precursor 1”)
(iv) Ac-Thr[α-D-M 6P]-Gly-Lys-(chelate)-Gly-Thr[α-D-M6P]-NH₂ (“glycopeptide precursor 2”)
   Where the vector is retinoic acid or a derivative thereof, the imaging moiety may be incorporated via the carboxyl group either directly or indirectly via a linker. The carboxylic group is the most preferred site for incorporation of an imaging moiety as it is not essential for binding to the M6P receptor. An example of a retinoic acid precursor compound with a linker and a chemical group capable of reacting with a source of an imaging moiety is:

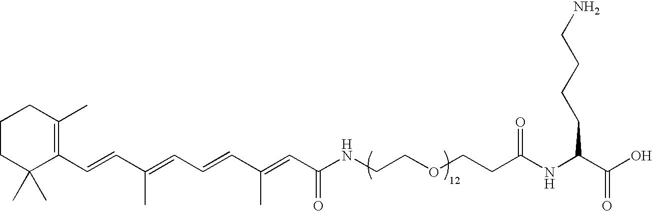

### “Retinoic Acid Precursor 1” (Synthesis Described in Example 9)

Retinoic acid is a lipophilic compound and as such where it is conjugated to a metal-complexing chelate to form a precursor compound, it should be conjugated to a hydrophilic metal-complexing chelate such as tetraamine.

In the case of combination vectors, any of the elements may be derivatised via suitable groups, as discussed above, that are available for derivatisation in order to form precursors.

In a preferred embodiment, the precursor of the invention is in sterile, apyrogenic form. The precursor can accordingly be used for the preparation of a pharmaceutical composition and is also suitable for inclusion as a component in a kit for the preparation of a pharmaceutical composition. These aspects are discussed in more detail below in relation to additional aspects of the invention.

In a further preferred embodiment the precursor of the invention is bound to a solid phase. The precursor is preferably supplied covalently attached to a solid support matrix in such a way that the labeling reaction results in simultaneous labeling and cleavage from the solid phase. The desired imaging agent product therefore forms in solution and any starting materials and impurities remain bound to the solid phase. As an example of such a system, precursors for solid phase electrophilic fluorination with ¹⁸F-fluoride are described in WO 03/002489, and precursors for solid phase nucleophilic fluorination with ¹⁸F-fluoride are described in WO 03/002157. A cartridge may be provided, preferably as part of a kit, which can be plugged into a suitably adapted automated synthesizer. The cartridge may contain, apart from the solid support-bound precursor, a column to remove unwanted fluoride ion, and an appropriate vessel connected so as to allow the reaction mixture to be evaporated and allow the product to be formulated as required.

A further aspect of the present invention is a precursor as defined in relation to the method of preparation of the imaging agent, wherein said chemical group preferably:
(i) comprises a chelator capable of complexing a metallic imaging moiety;
(ii) comprises an organometallic derivative such as a trialkylstannane or a trialkylsilane;
(iii) comprises a derivative containing an alkyl halide, alkyl tosylate or alkyl mesylate for nucleophilic substitution; or,
(iv) comprises a derivative which alkylates thiol-containing compounds to give a thioether-containing product.

In a further aspect, the present invention provides a pharmaceutical composition which comprises the imaging agent as described above, together with a biocompatible carrier, in a form suitable for mammalian administration. Preferably, the pharmaceutical composition is a radiopharmaceutical composition.

The “biocompatible carrier” is a fluid, especially a liquid, in which the imaging agent is suspended or dissolved, such that the composition is physiologically tolerable, i.e. can be administered to the mammalian body without toxicity or undue discomfort. The biocompatible carrier medium is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). The biocompatible carrier medium may also comprise biocompatible organic solvents such as ethanol. Such organic solvents are useful to solubilise more lipophilic compounds or formulations. Preferably the biocompatible carrier medium is pyrogen-free water for injection, isotonic saline or an aqueous ethanol solution. The pH of the biocompatible carrier medium for intravenous injection is suitably in the range 4.0 to 10.5.

Such pharmaceutical compositions are suitably supplied in either a container which is provided with a seal which is suitable for single or multiple puncturing with a hypodermic needle (e.g. a crimped-on septum seal closure) whilst maintaining sterile integrity. Such containers may contain single or multiple patient doses. Preferred multiple dose containers comprise a single bulk vial (e.g. of 10 to 30 cm³ volume) which contains multiple patient doses, whereby single patient doses can thus be withdrawn into clinical grade syringes at various time intervals during the viable lifetime of the preparation to suit the clinical situation. Pre-filled syringes are designed to contain a single human dose, or “unit dose” and are therefore preferably a disposable or other syringe suitable for clinical use. Where the pharmaceutical composition is a radiopharmaceutical composition, the pre-filled syringe may optionally be provided with a syringe shield to protect the operator from radioactive dose. Suitable such radiopharmaceutical syringe shields are known in the art and preferably comprise either lead or tungsten.

The pharmaceutical composition of the present invention may be prepared from a kits, as is described in a further aspect of the invention, below. Alternatively, the pharmaceutical composition may be prepared under aseptic manufacture conditions to give the desired sterile product. The pharmaceutical composition may also be prepared under non-sterile conditions, followed by terminal sterilisation using e.g. gamma-irradiation, autoclaving, dry heat or chemical treatment (e.g. with ethylene oxide). Preferably, the pharmaceutical composition of the present invention is prepared from a kit.

As described above in relation to the imaging agent of the invention, for radiopharmaceutical compositions the most preferred radioactive imaging moieties of the invention are ^{99m}Tc, ¹²³I and ¹⁸F.

In a yet further aspect, the present invention provides kits for the preparation of the pharmaceutical compositions of the invention. Such kits comprise a suitable precursor of the invention, preferably in sterile non-pyrogenic form, so that reaction with a sterile source of an imaging moiety gives the desired pharmaceutical composition with the minimum number of manipulations. Such considerations are particularly important for radiopharmaceuticals, especially where the radioisotope has a relatively short half-life, and for ease of handling and hence reduced radiation dose for the radiopharmacist. Hence, the reaction medium for reconstitution of such kits is preferably a “biocompatible carrier” as defined above, and is most preferably aqueous.

Suitable kit containers comprise a sealed container which permits maintenance of sterile integrity and/or radioactive safety, plus optionally an inert headspace gas (e.g. nitrogen or argon), whilst permitting addition and withdrawal of solutions by syringe. A preferred such container is a septum-sealed vial, wherein the gas-tight closure is crimped on with an overseal (typically of aluminium). Such containers have the additional advantage that the closure can withstand vacuum if desired e.g. to change the headspace gas or degas solutions.

Preferred aspects of the precursor when employed in the kit are as described above in relation to the method of synthesis of the imaging agents of the invention. The precursors for use in the kit may be employed under aseptic manufacture conditions to give the desired sterile, non-pyrogenic material. The precursors may also be employed under non-sterile conditions, followed by terminal sterilisation using e.g. gamma-irradiation, autoclaving, dry heat or chemical treatment (e.g. with ethylene oxide). Preferably, the precursors are employed in sterile, non-pyrogenic form. Most preferably the sterile, non-pyrogenic precursors are employed in the sealed container as described above.

The precursor of the kit is preferably supplied covalently attached to a solid support matrix as described above in relation to the method of synthesis of the imaging agents of the invention.

For ^{99m}Tc, the kit is preferably lyophilised and is designed to be reconstituted with sterile ^{99m}Tc-pertechnetate (TcO₄—) from a ^{99m}Tc radioisotope generator to give a solution suitable for human administration without further manipulation. Suitable kits comprise a container (e.g. a septum-sealed vial) containing the uncomplexed chelating agent, together with a pharmaceutically acceptable reducing agent such as sodium dithionite, sodium bisulphite, ascorbic acid, formamidine sulphinic acid, stannous ion, Fe(II) or Cu(I); together with at least one salt of a weak organic acid with a biocompatible cation. By the term “biocompatible cation” is meant a positively charged counterion which forms a salt with an ionised, negatively charged group, where said positively charged counterion is also non-toxic and hence suitable for administration to the mammalian body, especially the human body. Examples of suitable biocompatible cations include: the alkali metals sodium or potassium; the alkaline earth metals calcium and magnesium; and the ammonium ion. Preferred biocompatible cations are sodium and potassium, most preferably sodium.

The kits for preparation of ^{99m}Tc imaging agents may optionally further comprise a second, weak organic acid or salt thereof with a biocompatible cation, which functions as a transchelator. The transchelator is a compound which reacts rapidly to form a weak complex with technetium, then is displaced by the chelator of the kit. This minimises the risk of formation of reduced hydrolysed technetium (RHT) due to rapid reduction of pertechnetate competing with technetium complexation. Suitable such transchelators are the weak organic acids and salts thereof described above, preferably tartrates, gluconates, glucoheptonates, benzoates, or phosphonates, preferably phosphonates, most especially diphosphonates. A preferred such transchelator is MDP, ie. methylenediphosphonic acid, or a salt thereof with a biocompatible cation.

As an alternative to use of a chelator in free form, the kit for preparation of ^{99m}Tc imaging agents may optionally contain a non-radioactive metal complex of the chelator which, upon addition of the technetium, undergoes transmetallation (i.e. ligand exchange) giving the desired product. Suitable such complexes for transmetallation are copper or zinc complexes.

The pharmaceutically acceptable reducing agent used in the kit is preferably a stannous salt such as stannous chloride, stannous fluoride or stannous tartrate, and may be in either anhydrous or hydrated form. The stannous salt is preferably stannous chloride or stannous fluoride.

The kits may optionally further comprise additional components such as a radioprotectant, antimicrobial preservative, pH-adjusting agent or filler.

By the term “radioprotectant” is meant a compound which inhibits degradation reactions, such as redox processes, by trapping highly-reactive free radicals, such as oxygen-containing free radicals arising from the radiolysis of water. The radioprotectants of the present invention are suitably chosen from: ascorbic acid, para-aminobenzoic acid (i.e. 4-aminobenzoic acid), gentisic acid (i.e. 2,5-dihydroxybenzoic acid) and salts thereof with a biocompatible cation. The “biocompatible cation” and preferred embodiments thereof are as described above.

By the term “antimicrobial preservative” is meant an agent which inhibits the growth of potentially harmful micro-organisms such as bacteria, yeasts or moulds. The antimicrobial preservative may also exhibit some bactericidal properties, depending on the dose. The main role of the antimicrobial preservative(s) of the present invention is to inhibit the growth of any such micro-organism in the pharmaceutical composition post-reconstitution, i.e. in the imaging agent product itself. The antimicrobial preservative may, however, also optionally be used to inhibit the growth of potentially harmful micro-organisms in one or more components of the kit prior to reconstitution. Suitable antimicrobial preservative(s) include: the parabens, i.e. methyl, ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds useful to ensure that the pH of the reconstituted kit is within acceptable limits (approximately pH 4.0 to 10.5) for human or mammalian administration. Suitable such pH-adjusting agents include pharmaceutically acceptable buffers, such as tricine, phosphate or TRIS [ie. tris(hydroxymethyl)aminomethane], and pharmaceutically acceptable bases such as sodium carbonate, sodium bicarbonate or mixtures thereof. When the conjugate is employed in acid salt form, the pH adjusting agent may optionally be provided in a separate vial or container, so that the user of the kit can adjust the pH as part of a multi-step procedure.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

The imaging agents of the invention are useful for in vivo imaging. Accordingly, in an additional aspect, the present invention provides an imaging agent of the invention for use in an in vivo diagnostic or imaging method, e.g. SPECT or PET. Preferably said method relates to the in vivo imaging of a condition in which the M6P receptor is upregulated and therefore has utility in the diagnosis of conditions associated with fibrosis such as liver fibrosis, congestive heart failure, glomerulosclerosis and respiratory failure. In a most preferred embodiment, said condition is liver fibrosis, where the M6P receptor is known to be upregulated on HSC and on liver parenchymal cells.

This aspect of the invention also provides a method for the in vivo diagnosis or imaging in a subject of a condition in which the M6P receptor is upregulated, comprising administration of the pharmaceutical composition of the invention, described above. Said subject is preferably a mammal and most preferably a human. In an alternative embodiment, this aspect of the invention furthermore provides for the use of the imaging agent of the invention for imaging in vivo in a subject of a condition in which the M6P receptor is upregulated wherein said subject is previously administered with the pharmaceutical composition of the invention. By “previously administered” is meant that the step involving the clinician, wherein the pharmaceutical is given to the patient e.g., intravenous injection, has already been carried out.

This aspect of the invention also encompasses use of the imaging agent of the invention for the manufacture of pharmaceutical for the diagnostic imaging in vivo of a condition in which the M6P receptor is upregulated.

In another further aspect the invention provides a method of monitoring the effect of treatment of a human or animal body with a drug to combat a condition in which the M6P receptor is upregulated, said method comprising administering to said body an imaging agent of the invention and detecting the uptake of said imaging agent, said administration and detection optionally but preferably being effected repeatedly, e.g. before, during and after treatment with said drug.

### BRIEF DESCRIPTION OF THE EXAMPLES

Example 1 describes the synthesis of the aa48-55 peptide fragment of IGF-II linked to a PEG linker (IGF compound 1).

Example 2 describes the synthesis of the protected tetraamine chelate compound, tetra-Boc-tetraamine NHS ester (protected chelate Z).

Example 3 describes the steps necessary to link chelate Z to the aa48-55 peptide fragment of IGF-II linked to a PEG linker in order to produce a precursor of the present invention suitable for labeling with ^{99m}Tc (IGF precursor 1).

Example 4 describes how to label IGF precursor 1 with ^{99m}Tc to form IGF imaging agent 1.

Example 5 describes the synthesis of the diaminedioxime chelate, chelate X (i.e. Formula Xb where G=C).

Example 6 describes the steps necessary to link chelate X to the aa48-55 peptide fragment of IGF-II linked to a PEG linker in order to produce a precursor of the present invention suitable for labeling with ^{99m}Tc (IGF precursor 2).

Example 7 describes how to label IGF precursor 2 with ^{99m}Tc to form IGF imaging agent 2. Example 8 describes the steps necessary to link an iodinated Bolton-Hunter group to the aa48-55 peptide fragment of IGF-II linked to a PEG linker in order to produce a non-radioactive version of a radioiodinated imaging agent of the present invention (non-radioactive IGF imaging agent 3).

Example 9 describes the synthesis of the retinoic acid derivative retinoyl-PEG-Lys (retinoic acid precursor 1).

Example 10 describes the steps necessary to link an iodinated Bolton-Hunter group to the retinoic acid derivative retinoyl-PEG-Lys in order to produce a non-radioactive version of a radioiodinated imaging agent of the present invention (non-radioactive retinoic acid imaging agent 1).

Example 11 provides the synthesis of 1231-labelled glycopeptide imaging agent 1.

### EXAMPLES

### List of Abbreviations Used in Examples

AcOH acetic acid
C18 chain of 18 carbon atoms
DCM dichloromethane
Dde dichlorodiphenyl dichloroethylene
DMF dimethylformamide
EMS ethyl methyl sulphide
FMoc 9-fluorenylmethoxycarbonyl
HATU O-(7-azabenzotriazol-1-yl)-N,N,N′,N′-tetramethyluronium hexafluorophosphate
HBTU O-Benzotriazol-1-yl-N,N,N′,N′-tetramethyluronium hexafluorophosphate
HPLC high-performance liquid chromatography
MBHA 4-methylbenzhydrylamine

### NHS N-hydroxysuccinimide

### NMM N-Methylmorpholine

OtBu t-butyl ester
PEG polyethylene glycol
Pmc 2,2,5,7,8-pentamethylchroman-6-sulfonyl
tBu t-butyl
TFA trifluoroacetic acid
TFE tetrafluoroethylene
TIS triisopropylsilane.

### Example 1

### Synthesis of IGF compound 1 [H-PEG(4)-diqlvcoloyl-Phe-Arg-Ser-Met-Asp-Leu-Ala-Leu-NH₂]

The peptidyl-resin H-Phe-Arg(Pmc)-Ser(tBu)-Met-Asp(OtBu)-Leu-Ala-Leu-R was assembled on an Applied Biosystems 433A peptide synthesizer using Fmoc/tBu strategy starting with 0.25 mmol Fmoc-Rink amide MBHA Resin. An excess of 1 mmol pre-activated amino acids, using HBTU, was applied in the coupling steps.

Fmoc-amino PEG diglycolic acid (1 mmol), HATU (1 mmol) and N-methylmorpholine (2 mmol) were dissolved in DMF and left for 5 minutes. The mixture was added to the peptidyl-resin from above and left to react over night. Final Fmoc-deprotection was accomplished using 20% piperidine in DMF.

The peptidyl-resin (0.05 mmol) was treated with a solution of 2.5% water, 2.5% EMS and 2.5% TIS in TFA (10 mL) for 2 hours. The resin was removed by filtration and the filtrate evaporated in vacuo. Diethyl ether was added to the residue. The resulting precipitate was washed with ether and air-dried.

Purification by preparative HPLC (gradient: 10-40% B over 40 min where A=H₂O/0.1% TFA and B=ACN/0.1% TFA, flow rate: 10 mL/min, column: Phenomenex Luna 5μ C18 (2) 250×21.20 mm, detection: UV 214 nm, product retention time: 31.5 min) of the crude material afforded 6 mg pure product. The pure product was analysed by analytical HPLC (gradient: 10-40% B over 10 min where A=H₂O/0.1% TFA and B=ACN/0.1% TFA, flow rate: 0.3 mL/min, column: Phenomenex Luna 3μ C18 (2) 50×2 mm, detection: UV 214 nm, product retention time: 7.89 min). Further product characterisation was carried out using mass spectrometry (MH⁺ calculated: 1241.7, MH⁺ found: 1247.7).

### Example 2

### Synthesis of (8-Amino-2-{[tert-butoxycarbonyl-(2-tert-carbonylamino-ethyl)-amino]-methyl}-octyl)-(2-tert-butyloxycarbonylamino-ethyl)-carbamic acid tert-butyl ester (protected chelate Z)

### Step (a): 2-(6-Chloro-hexyloxy)tetrahydropyran

6-Chlorohexanol (6.85 g, 10 mmol) and p-toluenesulphonic acid (500 mg), were dissolved in dry ether (75 ml) and cooled to 0-5° C. in an ice bath. Dihydropyran (4.3 g, 10 mmol) in dry ether (25 ml) was added dropwise with constant stirring over a 30 minute period. After complete addition, the cooling bath was removed and stirring continued for 16 hours. The solution was extracted with water (50 ml×2), dried (MgSO₄), filtered and the solvent evaporated under reduced pressure to leave a pale yellow oil. This oil was shown by ¹³C NMR spectroscopy to be sufficiently pure to be used without purification in the subsequent reactions. Yield 10.1 g (91%).

¹H NMR (CDCl₃): δ 1.30-1.71 (14H, m, CH₂×7), 3.24-3.32 (1H 3.41-3.48 (3H, m CH and CH₂), 3.60-3.67 (1H, m, CH), 3.72-3.82 (1H, bm, CH), 4.44-4.49 (1H, bm, OCHO).

### Step (b): 2-[6-(Tetrahyhydro-pyran-2-yloxy)-hexyl]-malonic acid diethyl ester

Sodium (1.13 g, 49 mmol) in small quantities was dissolved in dry ethanol (100 ml) with constant stirring under a blanket of dry nitrogen. Diethyl malonate (8.0 g, 50 mmol) was added in one portion and the solution heated at 60° C. for 1 hour. 2-(6-Chloro-hexyloxy)-tetrahydropyran (9.3 g, 42.2 mmol) was added in one portion and the temperature raised to 75-80° C. and maintained at this level for 24 hours. After cooling, the inorganic solid was removed by filtration and solvent evaporated from the filtrate. The residue was dissolved in CH₂Cl₂ (50 ml), extracted with water (30 ml×2), dried (MgSO₄) filtered and volatiles removed to leave a pale yellow oil. This oil was subject to chromatography on silica gel using pet ether 40:60/ether (200:40) as the eluent. The required product eluted with an r_{f}=0.15 and was isolated as a colourless oil. Yield 8.7 g, (60%).

¹H NMR (CDCl₃): δ 1.10-1.25 (14H, m, CH₃×2, CH₂×4), 1.36-1.50 (6H, bm, CH₂×3), 1.70-1.81 (2H, bm, CH₂), 3.17-3.28 (2H, m, CH₂), 3.56-3.66 (1H, m, CH), 3.70-3.80 (1H, m, OCH), 4.04-4.16 (4H, m, OCH₂×2), 4.03-4.08 (1H, m, OCHO).

### Step (c): N,N′-Bis-(2-amino-ethyl)-2-[6-(tetrahydro-pyran-2-yloxy)-hexyl]-malonamide

2-[6-(Tetrahyhydro-pyran-2-yloxy)-hexyl]-malonic acid diethyl ester (5.1 g, 14.8 mmol) was dissolved in 1,2-diaminoethane (10 g, 167 mmol) and stirred at room temperature for 16 hours. Volatiles were removed in vacuo (40-50° C. at 0.01 mm Hg) to leave a pale green viscous residue which was subjected to column chromatography eluting with CH₂Cl₂/MeOH/NH₄OH (50:50:5). The title compound eluted with an r_{f} 0.2 and was collected as a pale green viscous oil which solidifies on standing. (Yield 3.9 g, 71%).

¹H NMR (CDCl₃): δ 1.15-1.28 (6H, bs, CH₂×3), 1.39-1.44 (6H, bm, CH₂×3), 1.69-1.74 (4H, bm, CH₂×2), 2.64 (4H, bs, NH₂×2), 2.73 4H, t, J=6 Hz, CH₂×2), 3.08-3.29 (6H, m, CH₂×3), 3.35-3.41 (1H, m CH), 3.55-3.63 (1H, m, CH), 3.70-3.78 (1H, m, CH), 4.43 (1H; bt, J=4 Hz, OCHO), 7.78 (2H, bt, J=5 Hz, OCNH×2)

IR (thin film) cm⁻¹: —3417, 3082, 2936, 2862, 1663, 1558, 1439, 1354, 1323, 1261, 1200, 1189, 1076, 1026, 956, 907, 867, 810.

### Step (d): N,N′-Bis(2-aminoethyl)-2-(6-hydroxy-hexyl)-malonamide

N,N′-Bis(2-aminoethyl)-2-[6-(tetrahydro-pyran-2-yloxy)-hexyl]-malonamide (3.9 g, 10.6 mmol), p-toluenesulphonic acid monohydrate (8.5 g, 3 mmol) and ethanol (50 ml) were heated under reflux at 70-75° C. for 16 hours. After cooling, concentrated ammonium hydroxide (0.880) was added dropwise until a permanent pH of 9 was achieved. The precipitated white solid was removed by filtration through Celite and the filter cake washed with ethanol (30 ml). The ethanol was removed under reduced pressure (15 mm Hg, 40° C.) to leave a semi-solid wax. This residue was subjected to chromatography on silica-gel eluting with CH₂Cl₂/MeOH/NH₄OH (100:50:10) and the title compound found to have an r_{f}=0.2. This product was collected and co-evaporated with ethanol (100 ml×3) to remove any residual water. A pale green viscous residue was obtained which solidifies on standing. (Yield 2.1 g, 69%).

¹H NMR (CD₃OD): δ 1.28-1.38 (6H, bs, CH₂×3), 1.46-1.55 (2H, bm, CH₂), 1.79-1.87 (2H, bm, CH₂), 2.73 (4H, t, J=6 Hz, H₂NCH₂×2), 3.13 (1H t, J=7 Hz, CH), 3.27 (4H, dt, J=6 and 2 Hz, HNCH₂×2), 3.53 (2H t, J=7 Hz OCH₂).

Mass spec (Fabs) m/e: —Calculated for C₁₃H₂₉N₄O₃ (M+H) 289 Found 289.

### Step (e): (2-tert-Butoxycarbonylamino-ethyl-2-{[tert-butoxycarbonyl-(2-tert-butoxycarbonylamino-ethyl)-amino]-methyl}-8-hydroxy-octyl)-carbonic acid tert-butyl ester

Under a blanket of dry nitrogen, neat borane-dimethylsulphide adduct (15 ml, 150 mmol) was added dropwise via a syringe to a stirred mixture of N,N′-bis(2-aminoethyl)-2-(6-hydroxyhexyl)malonamide (2.1 g, 7.3 mmol) in dioxane (50 ml). After complete addition, the mixture was heated gently under reflux at 110° C. for 5 days. During this period some white solid remained. After cooling the volatiles were removed under reduced pressure to leave a white solid to which methanol (50 ml) was added dropwise giving a colourless solution. This solution was heated under reflux for 3 hours, cooled, conc. HCl (5 ml) added and heating continued under reflux at 70-75° C. for 48 hours. The solvent was removed to leave a viscous green residue which was co-evaporated with methanol (100 ml×3) to leave a pale green solid. This solid was redissolved in dry methanol and anhydrous potassium carbonate (4.0 g, 30 mmol) added followed by di-tert-butyl dicarbonate (7.0 g, 32 mmol). The mixture was stirred at room temperature for 48 hours. The inorganic solid was removed by filtration through Celite and solvent evaporated from the filtrate to leave a viscous residue. This residue was mixed with water (50 ml) and extracted with CH₂Cl₂ (50 ml×3). The organic fractions were combined, dried (MgSO₄), filtered and the solvent evaporated to leave a pale yellow residue.

Note: At this point it is convenient to monitor the reaction by ¹³C NMR.

The residue was subjected to chromatography on silica gel using CH₂Cl₂/MeOH (95:5) as eluent. The title compound eluted with an r_{f}=0.41 and was isolated as a colourless viscous oil (Yield 2.5 g, 52%).

¹³C NMR (CDCl₃): δ 25.6 (CH₂), 26.4 (CH₂), 28.4 (CH₃×12), 29.8 (CH₂×2), 32.6 (CH₂), 36.5 (very broad, CH), 39.2 (NCH₂×2, adjacent CH), 46.9 (broad singlet, HNCH₂×2), 50.0 (broad singlet, NCH₂×2), 62.4 (HOCH₂), 79.0 (OC×2), 79.9 (OC×2), 156.4 (broad singlet C═O×4)

¹H NMR (CDCl₃): δ 1.05-1.18 (8H, bs, CH₂×4), 1.27 (18h, s, CH₃×6, t-butyl), 1.31 (18H, s, CH₃×6, t-butyl), 1.41 (2H, m, CH₂), 1.81 (1H bs, CH), 2.63 (1H, bs, OH), 2.98 (4H, bs, NCH₂×2), 3.11 (8H, bs, NCH₂×4), 3.44 (2H, t, J=8 Hz, CH₂O), 5.2 (2H, bs, NH×2)

Mass Spec (Fabs) m/e:—Calculated for C₃₃H₆₅N₄O₉ (M+H) 661 Found 661.

### Step (f): Toluene-4-sulfonic acid 8-[tert-butoxycarbonyl-(2-tert-butoxycarbonylamino-ethyl-amino]-7-{[tert-butoxycarbonyl-(2-tert-butoxycarbonylamino-ethyl)-amino]-methyl}-octyl Ester

(2-tert-Butoxycarbonylamino-ethyl-2-{[tert-butoxycarbonyl-(2-tert-butoxycarbonylaminoethyl)amino]-methyl}-8-hydroxyoctyl)-carbonic acid tert-butyl ester (2.52 g, 3.82 mmol), p-toluenesulfonyl chloride (1.0 g, 5.2 mmol), triethylamine (1.3 g, 12.8 mmol) and CH₂Cl₂ (30 ml) were stirred at room temperature with the slow evaporation of solvent. The reaction was monitored by carbon NMR and after 3 days little starting material remained. The reaction volume was made up to 30 ml with CH₂Cl₂, extracted with water (50 ml×3), dried (MgSO₄), filtered and the solvent evaporated to leave a brown residue. This residue was subjected to chromatography on silica gel using CH₂Cl₂/MeOH (100:5) as eluent. The first compound to elute was unreacted tosyl chloride with an r_{f}=0.95. The title compound eluted with an r_{f}=0.2 and was isolated as a pale yellow viscous oil. Yield (1.20 g, 39%).

¹H NMR (CDCl₃): δ 1.16 (8H, bs, CH₂×4), 1.35 (18H, s, CH₃×6), 1.39 (18H, s, CH₃×6), 1.88 (1H, bs, CH), 2.38 (3H, s, CH₃ Tosyl), 3.10-3.12 (4H, bs, NCH₂×2), 3.19 (8H, bs, NCH₂×4), 3.93 (2H, t, J=7 Hz, CH₂OTs), 5.0 (1H, bs, NH), 5.08 (1H, bs, NH), 7.29 (2H, d, J=8 Hz, CH×2, Ar), 7.72 (2H, d, J=8 Hz CH×2, Ar)

Mass Spec (Fabs) m/e:—Calculated for C₄₀H₇₁N₄O₁₁S (M+H) 815 Found 815.

### Step (a): (8-Azido-2-{[tert-butoxycarbonyl-(2-tert-carbonylamino-ethyl)-amino]-methyl}-octyl)-(2-tert-butyloxycarbonylamino-ethyl)-carbamic acid tert-butyl ester

Toluene-4-sulfonic acid 8-[tert-butoxycarbonyl-(2-tert-butoxycarbonylaminoethyl-amino]-7-{[tert-butoxycarbonyl-(2-tert-butoxycarbonylaminoethyl)amino]methyl}-octyl ester (1.105 g, 1.36 mmol), sodium azide (350 mg, 5.4 mmol) and methanol (10 ml) were heated under reflux at 70-75° C. for 16 hours. After cooling, methanol was removed at room temperature under reduced pressure until about 1-2 ml remained. This residue was diluted with water (25 ml) and extracted with CH₂Cl₂ (25 ml×4). The organic extracts were combined, dried (MgSO₄), filtered and volatiles evaporated at room temperature (Note: Azides are potentially explosive and this step should be carried out behind a safety shield) to leave a pale yellow viscous residue which was the desired compound in a pure state. (Yield 820 mg, 88%).

¹H NMR (CDCl₃): δ 1.16 (8H, bs, CH₂×4), 1.29 (18H, s CH₃×6), 1.33 (18H, s, CH₃×6), 1.47 (2H, bt, J=6.5 Hz CH₂ adjacent CH), 1.86 (1H, bs, CH), 2.95-3.05 (4H, bs, NCH₂×2), 3.05-3.20 (10H, bs, NCH₂×4 and CH₂N₃), 5.09 (2H, bs, NH×2)

IR (thin film) cm⁻¹:—3350, 2974, 2932, 2860, 2097 (Strong band N₃), 1694, 1520, 1470, 1418, 1391, 1366, 1250, 1167, 1069, 870, 777.

### Step (h): (8-Amino-2-{[tert-butoxycarbonyl-(2-tert-carbonylamino-ethyl)-amino]-methyl}-octyl)-(2-tert-butloxycarbonylamino-ethyl)-carbamic acid tert-butyl ester (Compound 2)

(8-Azido-2-{[tert-butoxycarbonyl-(2-tert-carbonylamino-ethyl)-amino]-methyl}-octyl)-(2-tert-butyloxycarbonylamino-ethyl)-carbamic acid tert-butyl ester (820 mg, 1.20 mmol), 10% palladium on charcoal (100 mg) and methanol (10 ml) were treated with hydrogen gas under a pressure of 30 atmospheres at room temperature for 16 hours. The solids were removed by filtration through Celite and the filter cake was washed with methanol (50 ml). Volatiles were removed from the filtrate to leave a viscous oil which was the desired material in a pure state. (Yield 700 mg, 89%).

¹H NMR (CDCl₃): δ 1.08 (8H, bs, CH₂×4), 1.23 (18H, s, CH₃×6), 1.27 (20H, bs, CH₃×6 and CH₂), 1.77 (1H, bs, CH), 2.40 (2H, bs, NH₂), 2.50 (2H, t, J=7 Hz, CH₂NH₂), 2.97 (4H, bm, NCH₂×2), 3.00-3.16 (8H, bm, NCH₂×4), 5.21 (1H, bs, NH), 5.30 (1H, bs, NH).

Mass Spec (Fabs) m/e:—Calculated for C₃₃H₆₆N₅O₈ (M+H) 660 Found 660.

### Example 3

### Synthesis of IGF precursor 1 [Tetraamine-PEG(4)-diglycoloyl-Phe-Arg-Ser-Met-Asp-Leu-Ala-Leu-NH₂]

The peptidyl-resin H-PEG(4)-diglycoloyl-Phe-Arg(Pmc)-Ser(tBu)-Met-Asp(OtBu)-Leu-Ala-Leu-R (0.05 mmol) was treated with a solution of tetra-Boc-tetramine NHS ester (0.05 mmol) and NMM (0.2 mmol) dissolved in DMF (5 mL) for 3 days. The reagents were removed by filtration and the resin washed with DMF and DCM.

The peptidyl-resin (0.05 mmol) was treated with a solution of 2.5% water, 2.5% EMS and 2.5% TIS in TFA (10 mL) for 2 hours. The resin was removed by filtration and the filtrate evaporated in vacuo. Diethyl ether was added to the residue. The resulting precipitate was washed with ether and air-dried.

Purification by preparative HPLC (gradient: 10-40% B over 40 min where A=H₂O/0.1% TFA and B=ACN/0.1% TFA, flow rate: 10 mL/min, column: Phenomenex Luna 5μ C18 (2) 250×21.20 mm, detection: UV 214 nm, product retention time: 25 min) of the crude material afforded 38 mg pure product. The pure product was analysed by analytical HPLC (gradient: 10-40% B over 10 min where A=H₂O/0.1% TFA and B=ACN/0.1% TFA, flow rate: 0.3 mL/min, column: Phenomenex Luna 3μ C18 (2) 50×2 mm, detection: UV 214 nm, product retention time: 7.05 min). Further product characterisation was carried out using mass spectrometry (MH⁺ calculated: 1441.8, M H⁺ found: 1441.7).

### Example 4

### ^{99m}Tc Radiolabelling of IGF Precursor 1 to Produce IGF Imaging Agent 1

^{99m}Tc complexes were prepared by adding the following to a nitrogen-purged P46 vial:
100 μg of IGF precursor 1 or 2 in MeOH,
0.5 ml Na₂CO₃/NaHCO₃ buffer (pH 9.2),
0.5 ml TcO₄— from a ^{99m}Tc generator,
0.1 ml SnCl₂/MDP solution,
(solution containing 10.2 mg SnCl₂ and 101 mg methylenediphosphonic acid in 100 ml N₂ purged saline).

ITLC (Instant thin layer chromatography) is used to determine the RCP. For IGF Precursor I, SG plates and a mobile phase of MeOH/(NH₄OAc 0.1M) 1:1 show RHT (reduced hydrolysed Tc) at the origin, pertechnetate at the solvent front and the technetium complex at an intermediate Rf.

The reaction mixture was analysed by reverse-phase HPLC (Xterra column RP18 3.5 μg, 150 mm×4.6 mm). HPLC conditions for IGF Precursor I:

Solvent A—0.07% ammonia in H₂O

### Solvent B—MeCN

0 min—90% solvent A, 10% solvent B
10 min—40% solvent A, 60% solvent B
15 min—40% solvent A, 60% solvent B
17 min—90% solvent A, 10% solvent B
20 min—90% solvent A, 10% solvent B

### Example 5

### Synthesis of chelate X [bis[N-(1,1-dimethyl-2-N-hydroxyimine propyl)-2-aminoethyl]-(2-aminoethyl)methane]

### (Step a): Preparation of tris(methyloxycarbonylmethyl)methane

3-(Methoxycarbonylmethylene)glutaric acid dimethylester (89 g, 267 mmol) in methanol (200 ml) was shaken with (10% palladium on charcoal: 50% water) (9 g) under an atmosphere of hydrogen gas (3.5 bar) for (30 h). The solution was filtered through kieselguhr and concentrated in vacuo to give 3-(methoxycarbonylmethyl)glutaric acid dimethylester as an oil, yield (84.9 g, 94%).

NMR ¹H(CDCl₃), δ 2.48 (6H, d, J=8 Hz, 3×CH₂), 2.78 (1H, hextet, J=8 Hz CH,) 3.7 (9H, s, 3×CH₃).

### (Step b): Amidation of Trimethylester with p-methoxy-benzylamine

Tris(methyloxycarbonylmethyl)methane [2 g, 8.4 mmol] was dissolved in p-methoxy-benzylamine (25 g, 178.6 mmol). The apparatus was set up for distillation and heated to 120° C. for 24 hrs under nitrogen flow. The progress of the reaction was monitored by the amount of methanol collected. The reaction mixture was cooled to ambient temperature and 30 ml of ethyl acetate was added, then the precipitated-triamide product stirred for 30 min. The triamide was isolated by filtration and the filter cake washed several times with sufficient amounts of ethyl acetate to remove excess p-methoxy-benzylamine. After drying 4.6 g, 100%, of a white powder was obtained. The highly insoluble product was used directly in the next step without further purification or characterisation.

### (Step c): Preparation of 1,1,1-tris[2-(p-methoxybenzylamino)ethyl]methane

To a 1000 ml 3-necked round bottomed flask cooled in a ice-water bath the triamide from step 2(a) (10 g, 17.89 mmol) is carefully added to 250 ml of 1M borane solution (3.5 g, 244.3 mmol) borane. After complete addition the ice-water bath is removed and the reaction mixture slowly heated to 60° C. The reaction mixture is stirred at 60° C. for 20 hrs. A sample of the reaction mixture (1 ml) was withdrawn, and mixed with 0.5 ml 5N HCl and left standing for 30 min. To the sample 0.5 ml of 50 NaOH was added, followed by 2 ml of water and the solution was stirred until all of the white precipitate dissolved. The solution was extracted with ether (5 ml) and evaporated. The residue was dissolved in acetonitrile at a concentration of 1 mg/ml and analysed by MS. If mono- and diamide (M+H/z=520 and 534) are seen in the MS spectrum, the reaction is not complete. To complete the reaction, a further 100 ml of 1M borane THF solution is added and the reaction mixture stirred for 6 more hrs at 60° C. and a new sample withdrawn following the previous sampling procedure. Further addition of the 1M borane in THF solution is continued as necessary until there is complete conversion to the triamine.

The reaction mixture is cooled to ambient temperature and 5N HCl is slowly added, [CARE: vigorous foam formation occurs!]. HCl was added until no more gas evolution is observed. The mixture was stirred for 30 min and then evaporated. The cake was suspended in aqueous NaOH solution (20-40%; 1:2 w/v) and stirred for 30 minutes. The mixture was then diluted with water (3 volumes). The mixture was then extracted with diethylether (2×150 ml) [CARE: do not use halogenated solvents]. The combined organic phases were then washed with water (1×200 ml), brine (150 ml) and dried over magnesium sulphate. Yield after evaporation: 7.6 g, 84% as oil.

NMR ¹H(CDCl₃), δ: 1.45 (6H, m, 3×CH₂; 1.54, (1H, septet, CH); 2.60 (6H, t, 3×CH₂N); 3.68 (6H, s, ArCH₂); 3.78 (9H, s, 3×CH₃O); 6.94 (6H, d, 6×Ar), 7.20 (6H, d, 6×Ar).

### (Step d): Preparation of 1,1,1-tris(2-aminoethyl)methane

1,1,1-tris[2-(p-methoxybenzylamino)ethyl]methane (20.0 gram, 0.036 mol) was dissolved in methanol (100 ml) and Pd(OH)₂ (5.0 gram) was added. The mixture was hydrogenated (3 bar, 100° C., in an autoclave) and stirred for 5 hours. Pd(OH)₂ was added in two more portions (2×5 gram) after 10 and 15 hours respectively.

The reaction mixture was filtered and the filtrate was washed with methanol. The combined organic phase was evaporated and the residue was distilled under vacuum (1×10⁻², 110° C.) to give 2.60 gram (50%) of 1,1,1-tris(2-aminoethyl)methane.

NMR ¹H(CDCl₃), δ 2.72 (6H, t, 3×CH₂N), 1.41 (H, septet, CH), 1.39 (6H, q, 3×CH₂).

NMR ¹³C(CDCl₃), δ 39.8 (CH₂NH₂), 38.2 (CH₂), 31.0 (CH).

### (Step e): Preparation of Chelate X (i.e. Formula Za where G=C)

To a solution of tris(2-aminoethyl)methane (4.047 g, 27.9 mmol) in dry ethanol (30 ml) was added potassium carbonate anhydrous (7.7 g, 55.8 mmol, 2 eq) at room temperature with vigorous stirring under a nitrogen atmosphere. A solution of 3-chloro-3-methyl-2-nitrosobutane (7.56 g, 55.8 mol, 2 eq) was dissolved in dry ethanol (100 ml) and 75 ml of this solution was dripped slowly into the reaction mixture. The reaction was followed by TLC on silica [plates run in dichloromethane, methanol, concentrated (0.88 sg) ammonia; 100/30/5 and the TLC plate developed by spraying with ninhydrin and heating]. The mono-, di- and tri-alkylated products were seen with RF's increasing in that order. Analytical HPLC was run using RPR reverse phase column in a gradient of 7.5-75% acetonitrile in 3% aqueous ammonia. The reaction was concentrated in vacuo to remove the ethanol and resuspended in water (110 ml). The aqueous slurry was extracted with ether (100 ml) to remove some of the trialkylated compound and lipophilic impurities leaving the mono and desired dialkylated product in the water layer. The aqueous solution was buffered with ammonium acetate (2 eq, 4.3 g, 55.8 mmol) to ensure good chromatography. The aqueous solution was stored at 4° C. overnight before purifying by automated preparative HPLC.

Yield (2.2 g, 6.4 mmol, 23%).

Mass spec; Positive ion 10 V cone voltage. Found: 344; calculated M+H=344.

NMR ¹H(CDCl₃), δ 1.24 (6H, s, 2×CH₃), 1.3 (6H, s, 2×CH₃), 1.25-1.75 (7H, m, 3×CH₂, CH), (3H, s, 2×CH₂), 2.58 (4H, m, CH₂N), 2.88 (2H, t CH₂N₂), 5.0 (6H s, NH₂, 2×NH, 2×OH).

NMR ¹³C((CD₃)₂SO), δ 9.0 (4×CH₃), 25.8 (2×CH₃), 31.0 2×CH₂, 34.6 CH₂, 56.8 2×CH₂N, 160.3; C═N.

HPLC conditions: flow rate 8 ml/min using a 25 mm PRP column

A=3% ammonia solution (sp.gr=0.88)/water; B=Acetonitrile

| Time | % B |
|---|---|
| 0 | 7.5 |
| 15 | 75.0 |
| 20 | 75.0 |
| 22 | 7.5 |
| 30 | 7.5 |

Load 3 ml of aqueous solution per run, and collect in a time window of 12.5-13.5 min.

### Example 6

### Synthesis of IGF Precursor 2 [chelate X-Glutaryl-PEG(4)-diglycoloyl-Phe-Arg-Ser-Met-Asp-Leu-Ala-Leu-NH₂]

The peptidyl-resin H-PEG(4)-diglycoloyl-Phe-Arg(Pmc)-Ser(tBu)-Met-Asp(OtBu)-Leu-Ala-Leu-R (0.05 mmol) was treated with a solution of Chelate X-glutaryl tetrafluorothiophenol ester (0.1 mmol) and NMM (0.2 mmol) dissolved in DMF (5 mL) for 3 days. The reagents were removed by filtration and the resin washed with DMF and DCM.

The peptidyl-resin (0.05 mmol) was treated with a solution of 2.5% water, 2.5% EMS and 2.5% TIS in TFA (10 mL) for 2 hours. The resin was removed by filtration and the filtrate evaporated in vacuo. Diethyl ether was added to the residue. The resulting precipitate was washed with ether and air-dried.

Purification by preparative HPLC (gradient: 20-40% B over 60 min where A=H₂O/0.1% TFA and B=ACN/0.1% TFA, flow rate: 10 mL/min, column: Phenomenex Luna 5μ C18 (2) 250×21.20 mm, detection: UV 214 nm, product retention time: 13 min) of the crude material afforded 35 mg pure product. The pure product was analysed by analytical HPLC (gradient: 10-50% B over 10 min where A=H₂O/0.1% TFA and B=ACN/0.1% TFA, flow rate: 0.3 mL/min, column: Phenomenex Luna 3μ C18 (2) 50×2 mm, detection: UV 214 nm, product retention time: 5.60 min). Further product characterisation was carried out using mass spectrometry (MH⁺ calculated: 1681.0, MH⁺ found: 1681.1).

### Example 7

### Radiolabelling of IGF Precursor 2 to Form IGF Imaging Agent 2

^{99m}Tc labelling is carried out using the method described in Example 4 above, but using the following HPLC and TLC conditions:

### HPLC.

### Solvent A—0.1% TFA in H₂O

### Solvent B—0.1% TFA in MeCN

1 min—90% solvent A, 10% solvent B
10 min—60% solvent A, 40% solvent B
15 min—60% solvent A, 40% solvent B
17 min—90% solvent A, 10% solvent B
20 min—90% solvent A, 10% solvent B
   TLC SG plates and a mobile phase of saline shows pertechnetate at the solvent front. Whatman 1 plates and a mobile phase of MeCN:H₂O 1:1 shows RHT at the origin.

### Example 8

### Synthesis of Non-Radioactive IGF Imaging agent 3 [3-(4-hydroxy-3-iodophenyl)propionate-PEG(4)-diglycoloyl-Phe-Arg-Ser-Met-Asp-Leu-Ala-Leu-NH₂]

The peptidyl-resin H-PEG(4)-diglycolyl-Phe-Arg(Pmc)-Ser(tBu)-Met-Asp(OtBu)-Leu-Ala-Leu-R (0.05 mmol) was treated with a solution of N-Succinimidyl-3-(4-hydroxy-3-iodophenyl) propionate (0.1 mmol) and NMM (0.2 mmol) dissolved in DMF (5 mL) for 3 days. The reagents were removed by filtration and the resin washed with DMF and DCM.

The peptidyl-resin (0.05 mmol) was treated with a solution of 2.5% water, 2.5% EMS and 2.5% TIS in TFA (10 mL) for 2 hours. The resin was removed by filtration and the filtrate evaporated in vacuo. Diethyl ether was added to the residue. The resulting precipitate was washed with ether and air-dried.

Purification by preparative HPLC (gradient: 10-50% B over 60 min where A=H₂O/0.1% TFA and B=ACN/0.1% TFA, flow rate: 10 mL/min, column: Phenomenex Luna 5μ C18 (2) 250×21.20 mm, detection: UV 214 nm, product retention time: 27 min) of the crude material afforded 5 mg pure product. The pure product was analysed by analytical HPLC (gradient: 10-50% B over 10 min where A=H₂O/0.1% TFA and B=ACN/0.1% TFA, flow rate: 0.3 mL/min, column: Phenomenex Luna 3μ C18 (2) 50×2 mm, detection: UV 214 nm, product retention time: 7.03 min). Further product characterisation was carried out using mass spectrometry (MH⁺ calculated: 1515.6, MH⁺ found: 1515.8).

### Example 9

### Synthesis of Retinoic Acid Precursor 1 [Retinoyl-PEG(12)-propionyl-Lys-OH]

Fmoc-Lys(Dde)-OH (Novabiochem, 0.4 mmol) was attached to an excess of 2-chlorotrityl chloride resin (Novabiochem) by standard procedure in dry dichloromethane using diisopropylethylamine (1.4 mmol) as base. The resin was washed with dichloromethane/methanol/diisopropylethylamine (17:2:1) mixture, dichloromethane and DMF. The Fmoc group was cleaved by standard piperidine treatment and Fmoc-aminoPEG(12)-propionic acid (Polypure AS, 0.6 mmol) was coupled using HATU (0.6 mmol) and diisopropylethylamine (1.2 mmol). Completion of the reaction was checked by standard Kaiser test. After cleavage of the Fmoc group retinoic acid (Fluka, 0.6 mmol) was coupled using the same coupling conditions. The Dde protecting group was removed by treatment with 2% hydrazine hydrate in DMF (3×2 min).

The resin from above (0.05 mmol) was treated with a solution of AcOH/TFE/DCM (2:2:6, 10 mL) for 45 min. The resin was removed by filtration, hexane (200 mL) added to the filtrate and the mixture evaporated in vacuo.

Purification by preparative HPLC (gradient: 30-80% B over 40 min where A=H₂O/0.1% TFA and B=ACN/0.1% TFA, flow rate: 10 mL/min, column: Phenomenex Luna 5μ C18 (2) 250×21.20 mm, detection: UV 214 nm, product retention time: 33 min) of the crude material afforded 17 mg pure product. The pure product was analysed by analytical HPLC (gradient: 30-80% B over 10 min where A=H₂O/0.1% TFA and B=ACN/0.1% TFA, flow rate: 0.3 mL/min, column: Phenomenex Luna 3μ C18 (2) 50×2 mm, detection: UV 214 nm, product retention time: 7.11 min). Further product characterisation was carried out using mass spectrometry (MH⁺ calculated: 1028.7, MH⁺ found: 1028.7).

### Example 10

### Synthesis of Non-Radioactive Retinoic Acid Imaging Agent 1 [Retinoyl-PEG(12)-propionyl-Lys(3-(4-hydroxy-3-iodophenyl)propionyl)-OH)]

The resin retinoyl-PEG(12)-propionyl-Lys-R (0.05 mmol) was treated with a solution of N-Succinimidyl-3-(4-hydroxy-3-iodophenyl) propionate (0.1 mmol) and NMM (0.2 mmol) dissolved in DMF (5 mL) for 20 hours. The reagents were removed by filtration and the resin washed with DMF and DCM.

The resin from above (0.05 mmol) was treated with a solution of AcOH/TFE/DCM (2:2:6, 10 mL) for 2 hour. The resin was removed by filtration, hexane (200 mL) added to the filtrate and the mixture evaporated in vacuo.

Purification by preparative HPLC (gradient: 50-100% B over 40 min where A=H₂O/0.1% TFA and B=ACN/0.1% TFA, flow rate: 10 mL/min, column: Phenomenex Luna 5μ C18 (2) 250×21.20 mm, detection: UV 214 nm, product retention time: 31 min) of the crude material afforded 17 mg pure product. The pure product was analysed by analytical HPLC (gradient: 50-95% B over 10 min where A=H₂O/0.1% TFA and B=ACN/0.1% TFA, flow rate: 0.3 mL/min, column: Phenomenex Luna 3μ C18 (2) 50×2 mm, detection: UV 214 nm, product retention time: 5.24 min). Further product characterisation was carried out using mass spectrometry (MH⁺ calculated: 1302.6, MH⁺ found: 1302.6).

### Example 11

### Synthesis of ¹²³I-labelled Glycopeptide Imaging Agent 1

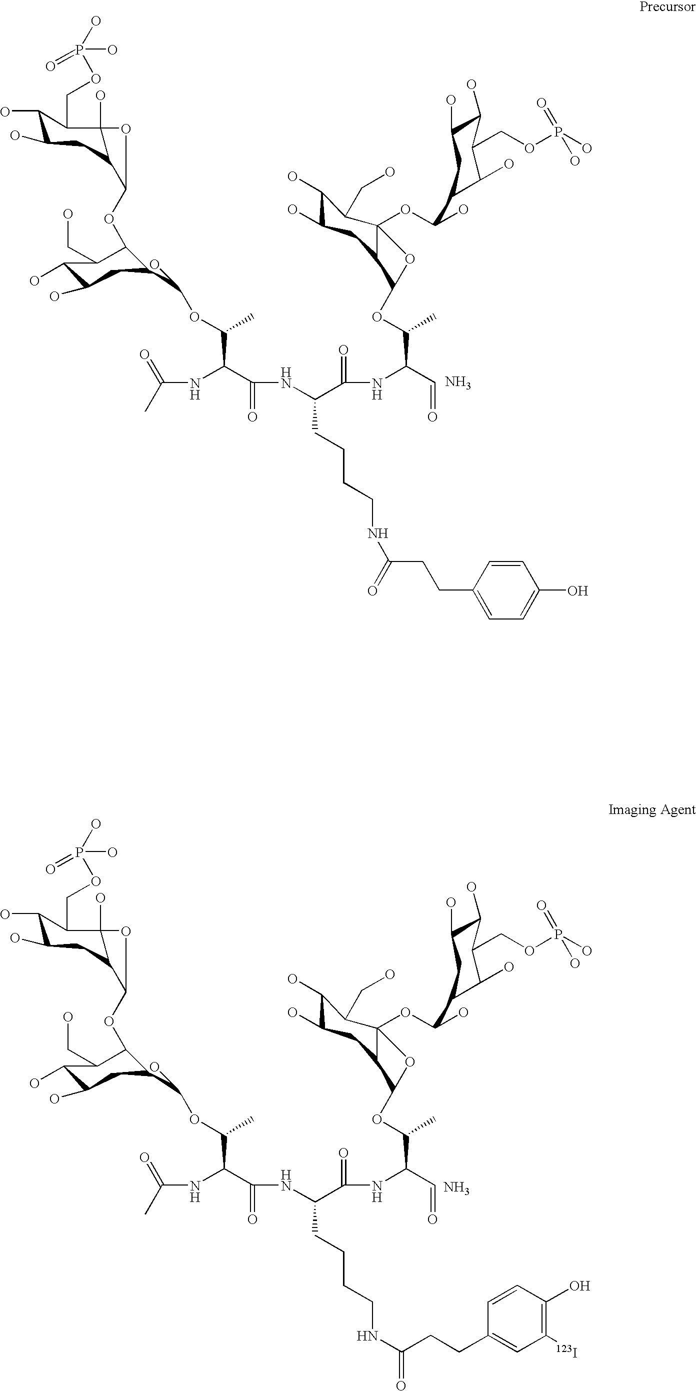

The precursor of Glycopeptide Imaging Agent 1 is shown above, and was radioiodinated as follows:

10 μl of ¹²⁷NaI (15 mg/100 ml in 0.01M NaOH) solution (containing 1×10⁻⁸ moles ¹²⁷NaI) was initially added to 200 μl ammonium acetate buffer (0.2 M, pH 4) in a microcentrifuge tube. The ¹²⁷NaI/NH₄OAc solution was then added to ¹²³I-NaI in 0.05 M NaOH (from GE Healthcare) containing 10 mCi (364 mCi/ml) of activity and the solution was mixed well. 5 μl of peracetic acid (36-40 wt % solution in acetic acid) was added to 5 ml H₂O, mixed well and 10 μl of the diluted solution was added to the ¹²³/¹²⁷I-NaI reaction mixture.

100 μl of precursor (0.74 mM solution in H₂O) was added to the reaction mixture, mixed with a pipette and the reaction was analysed after 5 minutes. The product was analysed or purified by HPLC using a Phenomenex Luna column 5u, C₁₈(2) 100 A, 150×4.6 mm.

### Solvent A—0.1% TFA in H₂O

Solvent B—0.1% TFA in acetonitrile
2 min—100% solvent A, 0% solvent B
20 min—50% solvent A, 50% solvent B
25 min—10% solvent A, 90% solvent B
35 min—10% solvent A, 90% solvent B
36 min—100% solvent A, 0% solvent B
43 min—100% solvent A, 0% solvent B
   The retention time of M6P Imaging Agent 1 under these conditions is 9.9 min.

What is claimed is:

1) An imaging agent comprising:
(i) a vector with affinity for the mannose-6-phosphate (M6P) receptor; and,
(ii) an imaging moiety
wherein the imaging moiety is present either as an integral part of the vector or the imaging moiety is conjugated to the vector via a suitable chemical group.

2) The imaging agent of claim 1 wherein said vector comprises at least one of the following:
(i) the 67 amino acid IGF-II sequence, or a fragment or analogue thereof;
(ii) mannose 6-phosphate (M6P);
(iii) a diphosphorylated glycopeptide; or
(iv) retinoic acid or a derivative thereof.

3) The imaging agent of claim 2 wherein said vector is the 67 amino acid IGF-II sequence, or is an 8-60 amino acid fragment or peptide analogue thereof selected from:
(i) a peptide comprising amino acid residues 48-55 (SEQ ID NO 2) or peptide analogues thereof;
(ii) a peptide comprising amino acid residues 8-28 (SEQ ID NO. 3) and 41-61 (SEQ ID NO. 4), or peptide analogues thereof, either joined directly or separated by a linker of formula -(L³)ₚ-;
(iii) a peptide comprising amino acid residues 8-67 (SEQ ID NO. 5), or peptide analogues thereof; and,
(iv) substitutions of various amino acid residues as follows: Phe26Ser (SEQ ID NO. 6); Phe19Ser (SEQ ID NO. 7); Glu12Lys (SEQ ID NO. 8); Tyr27Leu (SEQ ID NO. 9)
wherein for -(L³)ₚ- each L³ is independently —CO—, —CR₂—, —CR═CR—, —C≡C—, —CR₂CO₂—, —CO₂CR₂—, —NR—, —NRCO—, —CONR—, —NR(C═O)NR—, —NR(C═S)NR—, —SO₂NR—, —NRSO₂—, —CR₂OCR₂—, —CR₂SCR₂—, —CR₂NRCR₂—, a C₄₋₈ cycloheteroalkylene group, a C₄₋₈ cycloalkylene group, a C₅₋₁₂ arylene group, a C₃₋₁₂ heteroarylene group, an amino acid residue, a polyalkyleneglycol, polylactic acid or polyglycolic acid moiety;
p is an integer of value 0 to 30;
each R group is independently H or C₁₋₁₀ alkyl, C₃₋₁₀ alkylaryl, C₂₋₁₀ alkoxyalkyl, C₁₋₁₀ hydroxyalkyl, C₁₋₁₀ fluoroalkyl, or 2 or more R groups, together with the atoms to which they are attached form a carbocyclic, heterocyclic, saturated or unsaturated ring.

4) The imaging agent of claim 2 wherein said vector comprises M6P and is selected from:

| | | |
|---|---|---|
| (i) | M6P(α1,2)-Man-O(CH₂)₈CO₂Me | (α1,2 linked dimannoside) |
| (ii) | M6P(α1,3)-Man-O(CH₂)₈CO₂Me | (α1,3 linked dimannoside) |
| (iii) | M6P(α1,6)-Man-O(CH₂)₈CO₂Me | (α1,6 linked dimannoside) |
| (iv) | M6P(α1,2)-Man(1α,2)-Man-O(CH₂)₈CO₂Me | (α1,2 linked trimannoside) |
| (v) | The biantennary oligomannoside: | |

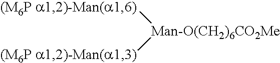

(v) The biantennary oligomannoside:
wherein Man is mannose.

5) The imaging agent of claim 2 wherein said vector is a diphosphorylated glycopeptide and is of Formula I: 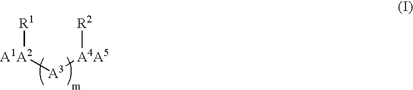
wherein
R¹ and R² are independently selected from:
(i) a natural L- or D-monosaccharide chosen from: glucose, mannose, galactose, fucose, rhammanose, N-acetylglucosamine, N-acetylgalactosaminyl, fructose and N-acetylneuraminic acid, or phosphorylated or sulphated versions thereof; or,
(ii) an oligosaccharide composed of monosaccharides selected from (i);
A¹ and A² are independently selected from the group consisting of —H, —OH, —NH₂, -acetyl, D- or L-amino acids, peptides, glycopeptides, peptidomimetics and oligonucleotides,
A² and A⁴ are independently selected from the group of D- or L-hydroxy amino acids, e.g. Ser, Thr, Hyl, Hyp, Tyr or D- or L-carboxamido amino acids, e.g. Asn and Gln, and
A³ is selected from the group of genetically encoded or non-encoded amino acids in their D- or L-form or peptidomimetics or nucleotides,
and wherein m is an integer between 1 and 30;
and wherein any residue in the linear sequence A¹-A⁵ may be covalently linked to form a cyclic compound.

6) The imaging agent of claim 5 wherein the diphosphorylated glycopeptide is
(i) Ac-Thr[α-D-M6P-(1,2)-α-D-mannose]-Lys (aminobenzamide)-Thr[α-D-M6P-(1,2)-α-D-mannose]-NH₂
(ii) Ac-Thr[α-D-M6P]-Gly-Lys-Gly-Thr[α-D-M6P]—NH₂

7) The imaging agent of claim 2 wherein said vector is retinoic acid, or an analogue thereof of Formula II: 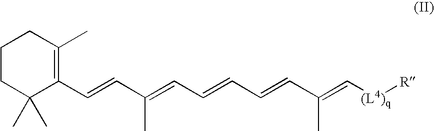
wherein L⁴, q and R″ are as defined for L³, p and R respectively in claim 3.

8) The imaging agent of claim 2 wherein said vector is a multivalent targeting vector combining two or more of the vectors of claim 2.

9) The imaging agent of claim 1 wherein said imaging moiety is selected from:
(i) a radioactive metal ion;
(ii) a paramagnetic metal ion;
(iii) a gamma-emitting radioactive halogen;
(iv) a positron-emitting radioactive non-metal;
(v) a hyperpolarised NMR-active nucleus;
(vi) a reporter suitable for in vivo optical imaging; and
(vii) a β-emitter suitable for intravascular detection.

10) The imaging agent of claim 9 wherein the imaging moiety is a radioactive metal ion.

11) The imaging agent of claim 10 wherein the radioactive metal ion is ^{99m}Tc.

12) The imaging agent of claim 9 wherein the imaging moiety is a gamma-emitting radioactive halogen.

13) The imaging agent of claim 12 wherein the gamma-emitting radioactive halogen is selected from ¹²³I or ¹³¹I.

14) The imaging agent of claim 9 wherein the imaging moiety is a positron-emitting radioactive non-metal.

15) The imaging agent of claim 14 wherein the positron-emitting radioactive non-metal is ¹⁸F.

16) A method for the preparation of the imaging agent of claim 1 comprising reaction of a precursor with a suitable source of an imaging moiety wherein said precursor comprises:
(i) a vector with affinity for M6P receptor as defined in said claim; and
(ii) a chemical group capable of reacting with a source of the imaging moiety so that the imaging moiety becomes attached to the compound to result in said imaging agent;
wherein said chemical group is either an integral part of said vector or is conjugated to said vector.

17) The method according to claim 16 wherein said chemical group:
(i) comprises a chelator capable of complexing a metallic imaging moiety;
(ii) comprises an organometallic derivative such as a trialkylstannane or a trialkylsilane;
(iii) comprises a derivative containing an alkyl halide, alkyl tosylate or alkyl mesylate for nucleophilic substitution;
(iv) comprises a derivative containing an aromatic ring activated towards nucleophilic or electrophilic substitution;
(v) comprises a derivative containing a functional group which undergoes facile alkylation;
(vi) comprises a derivative which alkylates thiol-containing compounds to give a thioether-containing product; or

18) The method according to claim 16 wherein said precursor is in sterile, apyrogenic form.

19) The method according to claim 16 wherein said precursor is bound to a solid phase.

20) A precursor as defined in the method of claim 16 wherein said chemical group:
(i) comprises a chelator capable of complexing a metallic imaging moiety;
(ii) comprises an organometallic derivative such as a trialkylstannane or a trialkylsilane;
(iii) comprises a derivative containing an alkyl halide, alkyl tosylate or alkyl mesylate for nucleophilic substitution;
(iv) comprises a derivative which alkylates thiol-containing compounds to give a thioether-containing product

21) A pharmaceutical composition comprising the imaging agent of claim 1, together with a biocompatible carrier in a form suitable for mammalian administration.

22) The pharmaceutical composition of claim 21 wherein said imaging agent comprises a radioactive imaging moiety.

23) The pharmaceutical composition of claim 22, which has a radioactive dose suitable for a single patient and is provided in a suitable syringe or container.

24) A kit for the preparation of the pharmaceutical composition of claim 21.

25) An imaging agent of claim 1 for use in an in vivo diagnostic or imaging method.

26) The imaging agent of claim 25 wherein said method relates to the in vivo imaging of a condition in which the M6P receptor is upregulated.

27) The imaging agent of claim 26 wherein the condition in which the M6P receptor is upregulated is a condition associated with fibrosis.

28) The imaging agent of claim 27 wherein said condition associated with fibrosis is liver fibrosis, congestive heart failure, glomerulosclerosis or respiratory failure.

29) The imaging agent of claim 28 wherein said condition associated with fibrosis is liver fibrosis.

30) The imaging agent of claim 29 wherein the M6P receptor is upregulated on liver parenchymal cells.

31) A method for the in vivo diagnosis or imaging in a subject of a condition in which the M6P receptor is upregulated, comprising administration of the pharmaceutical composition of claim 21.

32) Use of the imaging agent of claim 1 for imaging in vivo in a subject of a condition in which the M6P receptor is upregulated wherein said subject is previously administered with a pharmaceutical composition comprising the imaging agent of said claim, together with a biocompatible carrier in a form suitable for mammalian administration.

33) Use of the imaging agent of claim 1 for the manufacture of a pharmaceutical for the imaging in vivo of a condition in which the M6P receptor is upregulated.

34) A method of monitoring the effect of treatment of a human or animal body with a drug to combat a condition in which the M6P receptor is upregulated, said method comprising administering to said body the imaging agent of claim 1 and detecting the uptake of said imaging agent.
